(19) United States
(12) Patent Application Publication
(10) Pub. No.: US 2013/0330274 A1
(43) Pub. Date: Dec. 12, 2013

(54) COMPOSITIONS AND METHODS FOR DETECTING AND TREATING CANCER

(71) Applicant: University of Virginia Patent Foundation d/b/a University of Virginia Licensing & Ventures Group, Charlottesville, VA (US)

(72) Inventors: Stuart S. BERR, Crozet, VA (US); Landon W. Locke, Pittsburgh, PA (US)

(73) Assignee: University of Virginia Patent Foundation d/b/a University of Virginia Licensing & Ventures Group, Charlottesville, VA (US)

(21) Appl. No.: 13/899,634

(22) Filed: May 22, 2013

Related U.S. Application Data
(60) Provisional application No. 61/650,120, filed on May 22, 2012.

(51) Int. Cl. A61K 51/12

(52) U.S. Cl. CPC A61K 51/1234
USPC 424/1.21; 435/40.5

(57) ABSTRACT

Macrophages within the tumor microenvironment, also called tumor associated macrophages (TAMs) have been shown to play a major role in the growth and spread of many types of cancer. Cancer cells produce cytokines that cause the macrophages to differentiate into an M2 subtype. We have designed a mannosylated liposome (MAN-LIPs) and successfully showed it to accumulate in TAMs in a mouse model of pulmonary adenocarcinoma. These liposomes are loaded with ⁶⁴Cu to allow tracking by PET imaging, and contain a fluorescent dye in the lipid bilayer permitting subsequent fluorescence microscopy. MAN-LIPs are a promising new vehicle for the delivery of imaging agents to lung TAMs. In addition to imaging, they hold the potential for delivery of therapeutic agents to the tumor microenvironment.

### CROSS REFERENCE TO RELATED APPLICATIONS

This application is entitled to priority pursuant to 35 U.S.C. §119(e) to U.S. provisional patent application No. 61/650,120, filed on May 22, 2012. The entire disclosure of the afore-mentioned patent application is incorporated herein by reference.

### BACKGROUND

Macrophages participate centrally in many pulmonary diseases. In addition to their elevated presence in the lungs of COPD and CF patients, evidence has emerged linking macrophages to the development/progression of lung tumors. Enhancing cell survival, promoting tissue remodeling, angiogenesis, and suppressing the antitumor adaptive immune response are all pro-tumor functions of tumor associated macrophages (TAMs). In animal studies, the depletion or blockade of TAMs into the tumor microenvironment (TME) has been shown to inhibit tumor growth and reduce tumor vessel density. Clinical data have shown that TAMs are present in high density in many human tumors, including lung, breast, skin, and prostate. Furthermore, their presence and density correlates with cancer invasion and decreased patient survival time. The macrophage (MØ) subtype strongly influences its interaction with the tumor. The M2 subtype is found to facilitate tumor growth, while the M1 subtype impedes it, although exceptions to this generalization have been reported. TAMs represent a unique target for cancer therapies.

As a result of these observations, methods for monitoring TAMs in vivo are currently being explored to help better understand their role in lung tumor promotion and for assessing macrophage-targeted therapies. Molecular imaging using agents targeted to TAMs offers a noninvasive and quantitative method for assessing the presence and density of these cells. Some progress has been made in this area. A dextran-coated magneto-fluorescent nanoparticle decorated with the amino acid glycine (CLIO-gly) was reported to accumulate in TAMs. It also was shown that the agent could successfully track the depletion of TAMs following the administration of liposomal clodronate. Another agent, poly(1-glutamic acid)-Gd-chelated p-aminobenzyl-diethylenetriaminepentaacetic acid (PG-Gd-NIR813), was reported to label TAMs in vivo. The mechanism of uptake of CLIO-gly and PG-Gd-NIR813 by TAMs is currently undetermined.

Liposomes have certain advantages over the solid core particles previously used, such as the ability to deliver imaging agents or biologically active drugs in their aqueous core or lipid bilayer. The coating of the liposomes can be designed to target a known surface receptor on TAMs increasing the prospect of cell uptake. TAMs have been shown in numerous human and mouse studies to overexpress surface scavenger receptors, such as the mannose receptor (CD206), which is important for the clearance of mannose-bearing serum glycoproteins released at sites of inflammation and viruses, fungi, and bacteria.

There is a long felt need in the art for compositions and methods useful detecting and identifying tumors. The present invention satisfies these needs

### SUMMARY OF THE INVENTION

M2 macrophages (tumor associated) express scavenger receptors (e.g., the mannose receptor) and factors that facilitate tissue and blood vessel growth, suppress T cell mediated anti-tumor activity, and express enzymes that can break down the extracellular matrix, thereby promoting metastasis. Clinical studies have demonstrated a correlation between the density of M2 macrophages and patient prognosis.

The present invention is based on the discovery disclosed herein for the use of a novel mannosylated liposome loaded with ⁶⁴Cu for PET for detecting, imaging, and measuring TAMs in vivo. In one aspect, the method is non-invasive. One of ordinary skill in the art will appreciate that the described compositions and methods can be modified and still be compatible with the practice of the invention, including, for example, changing the imaging agent, changing the general components of the liposome (as long as they are still compatible with mannose and/or with imaging TAMs), etc. The present approach for monitoring TAMs by PET imaging has two significant advantages over fluorescently-labeled iron oxide nanoparticles designed for detection by MRI and optical techniques. First, the targeted liposomes are radiolabeled and thus can be tracked using PET imaging, which possesses a higher intrinsic sensitivity (10⁻⁸ to 10⁻⁹ mol/L for PET compared to 10⁻³ to 10⁻⁵ mol/L for MRI). Second, liposomes provide a flexible platform for delivering both hydrophobic and hydrophilic cargo. In the present application the aqueous interior core of liposomes was utilized to encapsulate a hydrophilic chelating agent to allow for remote labeling of the PET radionuclide, ⁶⁴Cu. This compartment of the liposome also could be used to encapsulate a wide array of hydrophilic drugs including cytotoxic agents that abolish TAMs through apoptosis or immunomodulatory agents that could trigger their reversal from a pro-tumoral M2 phenotype to a tumoricidal M1 phenotype.

The present invention further relates to selective cellular targeting for the purpose of locating tumor associated macrophages to help locate and image tumors. Therefore, the present compositions and methods are useful for delivering imaging agents to specific cells.

Mannosylated liposomes of the present invention are useful for both diagnostic imaging and delivery of therapeutic agents to the tumor microenvironment. In one aspect, the compositions and methods of the invention are useful for detecting, identifying, diagnosing, and treating cancer. In one aspect, the cancer is selected from the group consisting of squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. In one aspect, the cancer is lung cancer. In one aspect, the cancer is adenocarcinoma, or sarcoma, or carcinoma. In one aspect, the cancer is metastatic.

In one embodiment, a mannosylated liposome of the invention is preferentially taken up by TAMs. In one aspect, the invention provides compositions and methods useful for detecting, identifying, diagnosing, and treating cancer by targeting and imaging TAMs. In one aspect, the TAMs can be quantified.

In one aspect, a liposome of the invention is about 200 nm in diameter. In one aspect, a liposome of the invention has a diameter ranging from about 100 nm to about 300 nm. In one aspect, a liposome of the invention is about 150 nm in diameter. In one aspect, a liposome of the invention is about 250 nm in diameter.

In one aspect, a liposome of the invention comprises DOTA and optionally at least one agent or drug, such as a therapeutic agent or drug. In one aspect, the surface of the liposome comprises at least one mannose moiety. In one aspect, the mannose moiety is Man3-DPPE. In one aspect, a mannosylated liposome has many mannose moieties.

In one embodiment, a liposome of the invention is prepared according to the following method with the following components: 18.8 mg/mL of L-α-Phosphatidylcholine, 4.2 mg/mL of cholesterol, and optionally 0.025 mg/mL of the lipophilic fluorescent probe 3,3′-Dioctadecyloxacarbocyanine Perchlorate. A fluorescent probe is added if there will be fluorescent imaging used later. The liposomes are made using dehydration-rehydration: the lipids and DiO are dissolved in chloroform, the solvent is evaporated, and the resultant thin-film hydrated with a 10 mM solution of chelating agent 1,4,7,10-tetra-azacyclododecane-1,4,7,10-tetraacetic acid (DOTA) in 10 mM 4-(2-Hydroxyethyl)-1-piperazine-Ethanesulfonic Acid (HEPES) buffer with 150 mM NaCl and a pH of 4 for 2 hours at 37° C. and overnight at 4° C. The liposome solution is freeze-thawed 5 times and then extruded consecutively 20 times through 1 μm, 600 nm, 400 nm and 200 nm polycarbonate membrane filters using a Lipex extruder with high-pressure nitrogen. The non-encapsulated DOTA is removed by dialysis using a Slide-A-Lyzer G2 dialysis cassette with a molecular weight cut-off of 10,000 against five-2 liters of HEPES buffer containing 150 mM NaCl (pH 7.4).

In one aspect, mannosylated encapsulated liposomes (MAN-LIPs) are prepared as described above with the inclusion of neoglycolipids synthesized from mannotriose (Man₃) and dipalmitoylphosphatidylethanolamine (DPPE) by reductive amination. The mannosylated phospholipid is added at a 1:20 MAN-DPPE to phosphatidylcholine (PC) molar ratio and dissolved in chloroform. The mean particle diameter is verified by a laser light scattering particle size analyzer. A schematic diagram of plain and MAN-LIPS is in FIG. 1. One of ordinary skill in the art will appreciate that the method and components can be modified as long as the liposome still functions as required herein.

In one aspect, a liposome of the invention can be labeled for imaging. In one aspect, the label is a radiolabel. In one aspect, remote loading is used to radiolabel DOTA-containing liposomes with a useful PET probe, such as ⁶⁴Cu (t_{1/2}=12.7 h), by utilizing a lipophilic transporter, such as hydroxyquinoline, to ferry ⁶⁴Cu to the liposome interior where it is more tightly chelated by the encapsulated DOTA. Copper loading of the liposomes is confirmed using size exclusion chromatography (SEC) to determine if the fluorescent dye DiO labeled liposomes eluted in the same fractions as the radioactive ⁶⁴Cu. One of ordinary skill in the art will appreciate that the method can be modified as long the result is about the same or better. In one aspect, the radioactive isotope is selected from the group consisting of ¹¹C, ¹³N, ¹⁵O, ⁶⁴Cu, ⁶²Cu, ¹²⁴I, ⁷⁶Br, ⁸²Rb and ⁶⁸Ga. In one aspect, the chelating agent is selected from the group consisting of DTPA, DO3A, DOTA, EDTA, TETA, EHPG, HBED, NOTA, DOTMA, TETMA, PDTA, TTHA, LICAM, HYNIC, and MECAM.

In one aspect, labeled mannosylated liposomes are administered to a test subject. In one aspect, imaging is performed to help detect and identify the location of TAMs following administration of the mannosylated liposomes of the invention, and in turn detect, identify, and diagnose cancer. In one aspect, using AMIDE software or a similar program, MAN-LIP uptake in tumor and remote tissue, liver, and muscle is quantified with PET using co-registered, resolution-matched MR images to guide the size and location of PET ROIs. In one aspect, the tumor is a lung tumor. Other tissue or organs can be examined as well. An increase in an area suspected of having a tumor, relative to a control, is an indication of the presence of a tumor.

The present invention further provides compositions and methods useful for treating cancer by targeting TAMs. In one aspect, the liposomes of the invention can be modified to kill TAMs by incorporating drugs, macromolecules, or therapeutic agents into a liposome of the invention. The incorporation can be internal or surface. Liposomes of the invention can be used to deliver hydrophobic or hydrophilic agents.

In one aspect, the compositions and methods of the invention are useful for targeting CD206 positive cells and for imaging CD206 positive cells.

In one aspect, targeting TAMs with a mannosylated liposome of the invention, including one comprising a therapeutic agent or anti-cancer drug, is useful for inhibiting tissue and blood vessel growth stimulated by local tumor cells, counteracting the suppression of T cell mediated anti-tumor activity by the cancer cells, and inhibiting metastasis.

In one aspect, a liposome of the invention can be labeled with more than one type of imaging agent to allow the liposome, or cells targeted by the liposome, to be imaged or tracked using more than one detection method. For example, both a radiolabel and a fluorescent label can be used at the same time.

The present invention further encompasses methods of making the liposomes of the invention and also encompasses liposomes made by the methods of the invention.

The present invention provides pharmaceutical compositions comprising labeled mannosylated liposomes. The liposomes can be administered to a subject using various techniques. The amount of liposome administered can vary and can depend on the age, sex, and health of the subject, as well as the type of cancer to be imaged. For example, liposomes can be administered at doses from about 0.1 to about 100 μmol total phospholipid. One of ordinary skill in the art can determine a dose to be used. The amount of label can very depending on the label used and the imaging technique used. For example, when using ⁶⁴Cu, the present application discloses that the liposome dose was 1.9 μmol total phospholipid labeled with 50-75 μCi (1.85-2.8 MBq) of ⁶⁴Cu in a total volume of 160 μL. In one aspect, 100 to 10,000 μCi is used. In another aspect, 500 to 1,000 μCi is used. In one aspect, 400-500 μCi is used.

The present application further encompasses the use of other techniques for detecting, locating, or quantifying TAMs as well as for combinations of techniques. In one aspect, a technique of the invention can be used to also detect a tumor where the TAMs are associated with the tumor. An additional imaging technique can be used as well, for example, PET coupled with fluorescence. Useful techniques, depending on the label or optional label used, etc., include, but are not limited to, fluorescence, positron emission tomography (PET), magnetic resonance imaging (MRI), single photon emission computed tomography (SPECT/CT), intravital laser scanning microscopy, endoscopy, and radiographic imaging. The present technique further encompasses the use of MRI in conjunction with PET. Useful detectable labels, depending on the technique or combination of imaging techniques used, include, but are not limited to, a radionuclide, a radiological contrast agent, a paramagnetic ion, a metal, a biological tag, a fluorescent label, a chemiluminescent label, an ultrasound contrast agent, and a photoactive agent.

Useful radionuclides of the invention include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁸⁶Y, ⁹⁰Y, ⁸⁹Zr, ^{94m}Tc, ⁹⁴Tc, ^{99m}Tc, ¹²⁰I, ¹²⁵I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters.

The present application describes compositions and methods for preparing and using the mannosylated liposomes of the invention. For example, the present invention provides a mannosylated-liposome for detecting a tumor associated macrophage. In one aspect, the tumor associated macrophage is in a subject. In one aspect, the mannosylated liposome comprises L-α-Phosphatidylcholine, cholesterol, optionally 3,3′-Dioctadecyloxacarbocyanine Perchlorate (DiO), 1,4,7,10-tetra-azacyclododecane-1,4,7,10-tetraacetic acid (DOTA), a detectable label, and optionally an additional therapeutic agent.

In one embodiment, the present invention provides methods for making a mannosylated-liposome for detecting a tumor-associated macrophage. In one aspect, the method comprises preparing an encapsulated liposome comprising L-α-Phosphatidylcholine, cholesterol and optionally 3,3′-Dioctadecyloxacarbocyanine Perchlorate (DiO) using a dehydration-rehydration process wherein 1,4,7,10-tetra-azacyclododecane-1,4,7,10-tetraacetic acid (DOTA) is added at the rehydration step, and non-encapsulated DOTA is then removed. The mannosylation is achieved by adding mannosylated phospholipid to the phosphatidylcholine and dissolving it in chloroform, wherein the mannosylated phospholipid is synthesized from mannotriose and dipalmitoylphosphatidylethanolamine (DPPE) by reductive amination, and then incorporating a detectable label and optionally an effective amount of an additional therapeutic agent. An additional therapeutic agent can include, for example, at least one of a chemotherapeutic agent, an antimicrobial, an anesthetic, an anti-inflammatory, etc.

The present application provides the unexpected discovery of increased contrast between the tumor area and the background (surrounding normal tissue) when using a mannosylated liposome of the invention versus other liposome such as plain liposomes or PEG liposomes. The contrast is higher when using the mannosylated liposomes disclosed herein because they clear from the blood faster than do other types of liposomes, such as PEG liposomes, used by others. Therefore, the present invention provides for both earlier imaging times/capabilities than other methods as well as for higher contrast for better imaging of the tumor and surrounding tissue demarcations.

In one embodiment, a mannosylated liposome of the invention provides tumor-to-tissue contrast ratios of at least about 2.0 as measured by fluorescence images or other techniques. In one aspect, a mannosylated liposome of the invention provides tumor-to-tissue contrast ratios of at least about 2.5. In another aspect, a mannosylated liposome of the invention provides tumor-to-tissue ratios of at least about 3.0. In yet another aspect, a mannosylated liposome of the invention provides tumor-to-tissue ratios of at least about 3.5. In yet another aspect, a mannosylated liposome of the invention provides tumor-to-tissue ratios of at least about 4.0. In a further aspect, a mannosylated liposome of the invention provides tumor-to-tissue ratios of at least about 4.5. In another aspect, a mannosylated liposome of the invention provides tumor-to-tissue ratios of at least about 5.5. In another aspect, a mannosylated liposome of the invention provides tumor-to-tissue ratios of at least about 6.0. In another aspect, a mannosylated liposome of the invention provides tumor-to-tissue ratios of at least about 6.5. In yet another aspect, a mannosylated liposome of the invention provides tumor-to-tissue ratios of at least about 7.0. In another aspect, a mannosylated liposome of the invention provides tumor-to-tissue ratios of at least about 7.5. In one aspect, the ratio is from about 2.0 to about 7.5. In one aspect, the tissue is adjacent non-tumor tissue. In another aspect, the tissue is a different normal tissue from the counterpart tumor tissue, such as spleen when a tumor is a lung tumor.

In one embodiment, a mannosylated liposome of the invention provides a tumor-to-tissue contrast ratio greater than that of a plain liposome or a PEG liposome used in the same way for imaging. In one aspect, the contrast is at least about 1.2 times greater than that of a plain liposome or a PEG liposome. In another aspect, the contrast is at least about 1.3 times greater than that of a plain liposome or a PEG liposome. In a further aspect, the contrast is at least about 1.4 times greater than that of a plain liposome or a PEG liposome. In a further aspect, the contrast is at least about 1.5 times greater than that of a plain liposome or a PEG liposome. In a further aspect, the contrast is at least about 1.7 times greater than that of a plain liposome or a PEG liposome. In a further aspect, the contrast is at least about 1.9 times greater than that of a plain liposome or a PEG liposome. In a further aspect, the contrast is at least about 2.0 times greater than that of a plain liposome or a PEG liposome. In a further aspect, the contrast is at least about 2.3 times greater than that of a plain liposome or a PEG liposome. In a further aspect, the contrast is at least about 2.4 times greater than that of a plain liposome or a PEG liposome. In a further aspect, the contrast is at least about 2.7 times greater than that of a plain liposome or a PEG liposome. In a further aspect, the contrast is at least about 3.0 times greater than that of a plain liposome or a PEG liposome. In a further aspect, the contrast is at least about 3.4 times greater than that of a plain liposome or a PEG liposome. In a further aspect, the contrast is at least about 4.0 times greater than that of a plain liposome or a PEG liposome. In a further aspect, the contrast is at least about 4.4 times greater than that of a plain liposome or a PEG liposome. In a further aspect, the contrast is at least about 4.9 times greater than that of a plain liposome or a PEG liposome. In a further aspect, the contrast is at least about 5.0 times greater than that of a plain liposome or a PEG liposome. In a further aspect, the contrast is at least about 5.5 times greater than that of a plain liposome or a PEG liposome. In a further aspect, the contrast is at least about 7.5 times greater than that of a plain liposome or a PEG liposome. In a further aspect, the contrast is at least about 10.0 times greater than that of a plain liposome or a PEG liposome. In one aspect, the range of improved contrast is from about 1.2 to about 10. In another aspect, the range of improved contrast is from about 1.5 to about 8. In one aspect, the range of improved contrast is from about 2.0 to about 6. In one aspect, the range of improved contrast is from about 3.0 to about 5.

The compositions and methods of the present invention are also useful for detecting and quantifying other cell types expressing mannose receptors, not just tumor associated macrophages.

The present invention provides at least one kit comprising the ingredients for making the labeled liposomes of the invention, an applicator for administration, and an instructional material for the use thereof.

Various aspects and embodiments of the invention are described in further detail below.

### BRIEF DESCRIPTION OF THE DRAWINGS

FIG. 1. A schematic diagram of liposomes. DOTA-containing plain (left) and Man₃ (right) liposomes allow for remote loading of the PET imaging agent, ⁶⁴Cu. The mean liposome diameter was 200 nm.

FIG. 2. Macrophage M2 polarization in lungs of a urethane-treated mouse 25 weeks after treatment. The H&E image shows a tumor boundary (T) with moderate immune cell infiltration in the stroma (A). Immunofluorescence staining of the tumor stroma outlined in the H&E image by the black dashed box is shown in (B). At the edge of the tumor (indicated by dashed white line), the majority of macrophages identified by F4/80 (red) also stain for CD206 (green) indicating M2 polarization.

FIG. 3. Representative spin echo MR images of a saline (top) and urethane injected (bottom) mouse 24 weeks following treatment. These scans were acquired approximately 30 min after gadolinium injection. Lungs tumors, as indicated by yellow arrows, are clearly visible in the urethane-treated mouse. In contrast, no lung tumors are detected in the saline-treated mouse.

FIG. 4. Representative in vivo images showing a lung tumor on coronal MRI (A) with enhanced ⁶⁴Cu-labeled Man₃-liposome uptake on PET 6 h after i.v. injection (B). PETMR image registration (C) verifies tumor localization of the PET signal. Ex vivo fluorescence image of the lung was obtained to assess DiO distribution. A photo shows the tumor (D) which exhibited higher DiO accumulation compared to non-tumor lung areas. The fluorescence image overlaid on the photo reveals a strong DiO-fluorescent signal within the tumor and minimal accumulation in non-tumor areas of the lung (FIG. 4E). Ex vivo PET maximum intensity projection image (F) showed focal ⁶⁴Cu signal in the area of the lung spatially corresponding to the tumor shown in the photograph.

FIG. 5. Confocal fluorescence microscopy revealed internalization of DiO-labeled Man₃₋ liposomes (green) by F4/80+ macrophages (red) within the tumor stroma 6 h after i.v. liposome injection. Cell nuclei are stained with DAPI (blue). (A) A co-localized confocal image shows the intracellular localization of Man₃₋ liposomes within TAMs. The enlarged view of the cell indicated by the yellow arrow is shown in (B), clearly shows a clustered distribution of liposomes consistent with storage in macrophage endosomal structures.

FIG. 6. Representative photos and fluorescent images from the liposome co-injection study. Images of excised lungs 6 h after the co-injection of Man₃ and plain liposomes (A) and Man₃ and PEG liposomes (B). Strong fluorescence signal associated with Man₃₋ and plain liposomes is localized to lung tumors (identified by white arrows on the photo). However, compared to Man₃-liposomes, plain liposomes exhibit a higher background signal. PEG liposomes show a diffuse lung distribution and consequently poor tumor contrast likely due to their enhanced blood circulation time.

FIG. 7. Tumor-to-tissue ratios measured from fluorescence images of harvested organs following the co-injection of MAN and plain liposomes (A) and MAN and PEG liposomes (B). MAN-LIPs exhibited a higher tumor-to-remote lung and tumor-to-spleen ratio compared to plain liposomes, while tumor-to-liver ratios were comparable. MAN-liposomes also exhibited a high tumor-to-remote lung ratio following co-injection with PEG liposomes, likely due to the slow rate of blood clearance of PEG liposomes. PEG liposomes also showed a reduced tumor-to-liver ratio, consistent with a lower rate of capture by the RES.

### DETAILED DESCRIPTION

### Abbreviations and Acronyms

BM—bone marrow

CD206—mannose receptor

CLIO-gly—dextran-coated magneto-fluorescent nanoparticle decorated with the amino acid glycine

CRD—carbohydrate recognition domain

DiD—1,1′-Dioctadecyl-3,3,3′,3′-Tetramethylindodicarbocyanine

DiO—3,3′-Dioctadecyloxacarbocyanine Perchlorate

DOTA—1,4,7,10-tetraazacyclododecane-1,4,7,10-tetraacetic acid (also referred to as 1,4,7,10-tetra-azacyclododecane-1,4,7,10-tetraacetic acid)

DPPE—dipalmitoylphosphatidylethanolamine

IFN—interferon

IL—interleukin

IP—intraperitoneal

MBq—megabecquerel

MØ—macrophage

M1—a subtype of macrophage found to impede tumor growth

M2—a subtype of macrophage found to facilitate tumor growth

Man₃—mannotriose

MAN-LIP—mannosylated encapsulated liposome

MEM—minimum essential medium

MMR—macrophage mannose receptor

PC—phosphatidylcholine

PEG—polyethyleneglycol

PET—positron emission tomography

PG-Gd-NIR813—poly(1-glutamic acid)-Gd-chelatedp-aminobenzyl-diethylenetriaminepentaacetic acid

PVE—partial volume effect

RC—recovery coefficient

ROI—region of interest

SEC—size exclusion chromatography

SPECT—single photon emission computed tomography

TAM—tumor-associated macrophages

TME—tumor microenvironment

### DEFINITIONS

In describing and claiming the invention, the following terminology will be used in accordance with the definitions set forth below.

The articles “a” and “an” are used herein to refer to one or to more than one (i.e. to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

As used herein, “adenocarcinoma” refers to a cancerous tumor as opposed to an “adenoma” which refers to a benign (non-cancerous) tumor made up of cells that form glands (collections of cells surrounding an empty space).

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the invention or a prodrug of a compound of the invention to a subject in need of treatment.

As used herein, the term “aerosol” refers to suspension in the air. In particular, aerosol refers to the particlization or atomization of a formulation of the invention and its suspension in the air.

As used herein, an “agonist” is a composition of matter which, when administered to a mammal such as a human, enhances or extends a biological activity attributable to the level or presence of a target compound or molecule of interest in the mammal.

The term “alterations in peptide structure” as used herein refers to changes including, but not limited to, changes in sequence, and post-translational modification.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

Amino acids have the following general structure:

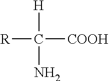

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

As used herein, the term “attach”, or “attachment”, or “attached”, or “attaching”, used herein interchangeably with “bind”, or “binding” or “binds' or “bound” refers to any physical relationship between molecules that results in forming a stable complex, such as a physical relationship between a ligand, such as a peptide or small molecule, with a “binding partner” or “receptor molecule.” The relationship may be mediated by physicochemical interactions including, but not limited to, a selective noncovalent association, ionic attraction, hydrogen bonding, covalent bonding, Van der Waals forces or hydrophobic attraction.

As used herein, the term “avidity” refers to a total binding strength of a ligand with a receptor molecule, such that the strength of an interaction comprises multiple independent binding interactions between partners, which can be derived from multiple low affinity interactions or a small number of high affinity interactions.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

As used herein, the term “biopsy tissue” refers to a sample of tissue that is removed from a subject for the purpose of determining if the sample contains cancerous tissue. In some embodiment, biopsy tissue is obtained because a subject is suspected of having cancer. The biopsy tissue is then examined for the presence or absence of cancer.

The term “cancer”, as used herein, is defined as proliferation of cells whose unique trait—loss of normal controls—results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer, and lung cancer.

As used herein, the term “carrier molecule” refers to any molecule that is chemically conjugated to a molecule of interest.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

By the term “contrast” in the context of comparing the ratios of tumor to tissue when using a mannosylated liposome of the invention relative to a liposome or a PEG liposome, does not mean that the ratio has to apply to both a liposome and a PEG liposome at the same time,

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

The term “elixir,” as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “invasive,” or “metastasis” as used herein, refers to any migration of cells, especially to invasive cancer cells or tumor cells. The term applies to normally invasive cells such as wound-healing fibroblasts and also to cells that migrate abnormally. Although the term is not to be limited by any mechanistic rationale, such cells are thought to migrate by defeating the body's means for keeping them sufficiently “in place” to function normally. Such cells are “invasive” if they migrate abnormally within a tissue or tumor, or escape the tissue, or invade other tissues.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor or target molecule.

A “receptor” or target molecule is a compound that specifically binds to a ligand.

A ligand or a receptor “specifically binds to” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

As used herein, the term “malignant” refers to having the properties of anaplasia, penetrance, such as into nearby areas or the vasculature, and metastasis.

The term “mass tag”, as used herein, means a chemical modification of a molecule, or more typically two such modifications of molecules such as peptides, that can be distinguished from another modification based on molecular mass, despite chemical identity.

The term “method of identifying peptides in a sample”, as used herein, refers to identifying small and large peptides, including proteins.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

As used herein, the term “peptide ligand” (or the word “ligand” in reference to a peptide) refers to a peptide or fragment of a protein that specifically binds to a molecule, such as a protein, carbohydrate, and the like. A receptor or binding partner of the peptide ligand can be essentially any type of molecule such as polypeptide, nucleic acid, carbohydrate, lipid, or any organic derived compound. Specific examples of ligands are peptide ligands of the present inventions.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

As used herein, the term “post surgical tumor tissue” refers to cancerous tissue (e.g., biopsy tissue) that has been removed from a subject (e.g., during surgery).

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties. As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, DC, p. 574).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. As used herein, the term “non-cancerous” in reference to a pancreatic cell refers to a cell demonstrating regulatable cell growth and functional physiology relative to its developmental stage and activity.

As used herein, a “subject in need thereof” is a patient, animal, mammal, or human, who will benefit from the method of this invention.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “tumor” refers to an abnormal mass of tissue that results from excessive cell division that is uncontrolled and progressive. It is also called a neoplasm. Tumors may be either benign (not cancerous) or malignant.

As used herein, the term “tumor cell”, as used herein, refers to any mass of cells that exhibits any uncontrolled growth patterns or altered physiology. Tumor cells may be derived from any tissue within an organism (e.g., a pancreatic ductal tumor cell). As used herein, the term “cancer” is a general term for more than 100 diseases that are characterized by an uncontrolled, abnormal growth of cells. Cancer cells can spread locally or can intravasate and spread via the bloodstream and lymphatic system to other parts of the body and form metastases. Cancer cells that spread are called “malignant.” As used herein, the terms “cancer” and “cancerous” in reference to a physiological condition in mammals is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

By the term “vaccine,” as used herein, is meant a composition which when inoculated into a subject has the effect of stimulating an immune response in the subject, which serves to fully or partially protect the subject against a condition, disease or its symptoms. In one aspect, the condition is conception. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### EMBODIMENTS

The liposomes of the invention may comprise chelators. The chelator is useful as a moiety to which an imaging agent can be added, such as ⁶⁴Cu.

The chelator of the invention can be selected from the group consisting of DTPA, DO3A, DOTA, EDTA, TETA, EHPG, HBED, NOTA, DOTMA, TETMA, PDTA, TTHA, LICAM, HYNIC, and MECAM.

DOTA has the structure:

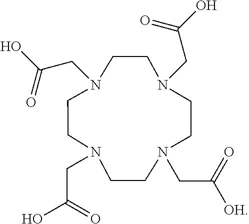 [SMILES: O=C(O)CN1CCN(CC(=O)O)CCN(CC(=O)O)CCN(CC(=O)O)CC1]

In one aspect, the imaging agent selected from the group consisting of a radionuclide, a radiological contrast agent, a paramagnetic ion, a metal, a biological tag, a fluorescent label, a chemiluminescent label, an ultrasound contrast agent and a photoactive agent. In one aspect, the imaging agent is a radionuclide. In one aspect, the radionuclide is selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, and other gamma-, beta-, or positron-emitters. In one aspect, the radionuclide is ⁶⁴Cu.

In one aspect, the method provides for the use of an imaging agent (detectable label) selected from the group consisting of a radionuclide, a radiological contrast agent, a paramagnetic ion, a metal, a biological tag, a fluorescent label, a chemiluminescent label, an ultrasound contrast agent and a photoactive agent. One of ordinary skill in the art will understand that the method of detection used will depend on the particular imaging agent used.

In one aspect, the invention further provides a method for detecting cancer, diagnosing cancer, monitoring the progression of cancer, or monitoring treatment of a cancer. In one aspect, the method is an imaging method.

The “macrophage mannose receptor” (MMR), as used herein, refers to a type 1 transmembrane protein, first identified in mammalian tissue macrophages and later in dendritic cells and a variety of endothelial and epithelial cells. Macrophages are central actors of the innate and adaptive immune responses. They are disseminated throughout most organs to protect against entry of infectious agents by internalizing and most of the time, killing them. Among the surface receptors present on macrophages, the mannose receptor recognizes a variety of molecular patterns generic to microorganisms. The MMR is composed of a single subunit with N- and O-linked glycosylations and consists of five domains: an N-terminal cysteine-rich region, which recognizes terminal sulfated sugar residues; a fibronectin type II domain with unclear function; a series of eight C-type, lectin-like carbohydrate recognition domains (CRDs) involved in Ca²⁺-dependent recognition of mannose, fucose, or N-acetylglucosamine residues on the envelop of pathogens or on endogenous glycoproteins with CRDs 4-8 showing affinity for ligands comparable with that of intact MR; a single transmembrane domain; and a 45 residue-long cytoplasmic tail that contains motifs critical for MR-mediated endocytosis and sorting in endosomes.

Modifications

The present invention further provides for the use of molecules such as polyethylene glycol (“PEG”) molecules as part of the complex. In one aspect, the PEG is about 20,000 m.w. or about less than about 20,000 m.w. In another aspect, the PEG is less than about 18, 000 m.w. In yet another aspect, the PEG is less that about 16,000 m.w. In a further aspect, the PEG is less than about 14,000 m.w. In a further aspect, the PEG is less than about 12,000 m.w. In a further aspect, the PEG is less than about 10,000 m.w. In a further aspect, the PEG is less than about 8,000 m.w. In a further aspect, the PEG is less than about 7,000 m.w. In a further aspect, the PEG is less than about 6,000 m.w. In a further aspect, the PEG is less than about 5,000 m.w. In a further aspect, the PEG is less than about 4,000 m.w. In a further aspect, the PEG is less than about 3,000 m.w. In a further aspect, the PEG is less than about 2,000 m.w. In a further aspect, the PEG is less than about 1,000 m.w. In a further aspect, the PEG is less than about 500 m.w.

In one aspect, the PEG is PEG5000.

Peptide Modification and Preparation

Peptide preparation is described in the Examples. It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the invention are not limited to products of any of the specific exemplary processes listed herein.

The invention includes the use of beta-alanine (also referred to as β-alanine, β-Ala, bA, and βA, having the structure:

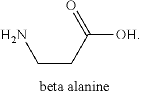

Sequences are provided herein which use the symbol “βA”, but in the Sequence Listing submitted herewith “βA” is provided as “Xaa” and reference in the text of the Sequence Listing indicates that Xaa is beta alanine

Peptides useful in the present invention, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C₄-,C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As discussed, modifications or optimizations of peptide ligands of the invention are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+-0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (O) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Diagnosing, Monitoring, and Treating Cancer

In one aspect, the invention provides in vivo methods and compositions for diagnosing a cancer. The methods include identifying a subject at risk for or suspected of having cancer; administering to a subject a diagnostic composition comprising a liposome complex of the invention conjugated to or comprising one or more imaging molecules, and imaging the imaging molecule within the subject using in vivo imaging. In some embodiments, the composition is administered via route selected from the group consisting of intradermal, subcutaneous, intraperitoneal, intravenous, intraarterial, oral, and gastric routes. In some embodiments, the in vivo imaging includes but is not limited to magnetic resonance imaging (MRI), intravital laser scanning microscopy, endoscopy, PET, SPECT/CT, and radiographic imaging.

The invention further provides for monitoring the progression of cancer, including during carcinogenesis.

In one embodiment, the present invention further provides compositions and methods for monitoring the progression or treatment of a cancer.

In another embodiment, the present invention provides methods for surgically removing a tumor(s). The methods include a) providing: i) a composition comprising a liposome complex of the invention for distinguishing a cancer ii) a subject known to have cancer; iii) an in vivo imaging device; and b) administering the composition to a subject; c) imaging TAMS to identify the cancer in vivo with the imaging device; and d) removing the tumor from the subject following detecting their location.

The present invention provides a method of treating a subject with cancer, comprising, a) providing: i) a subject in need of treatment; ii) a pharmaceutical composition comprising a liposome complex of the invention, wherein the liposome complex binds to TAMs; and b) administering the treatment composition to the subject. In some embodiments, the pharmaceutical composition further comprises a therapeutic agent. In some embodiments, the therapeutic agent is selected from the group consisting of at least one fusion protein, a toxin, and a drug, or a combination thereof.

Indeed, various types of cancer are contemplated for use with the detection methods of the present inventions including, but not limited to lung cancer, bladder cancer, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer; cervical cancer, endometrial cancer, uterine cancer, pancreatic cancer, colon cancer, colorectal, gastric cancer, kidney cancer, bladder cancer, lymphoma cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, white blood cell cancer (e.g., lymphoma, leukemia, etc.), hereditary non-polyposis cancer (HNPC), colitis-associated cancer, etc. Cancers are further exemplified by sarcomas (such as osteosarcoma and Kaposi's sarcoma).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.). Such nucleic acids can be incorporated into a liposome of the invention.

A comparison of the levels and location in the test subject is made with the levels and location of the imaging agent from an otherwise identical location from an unaffected subject or with an unaffected area of the test subject. A higher level or different location of the imaging agent in the test subject compared with the level or location of the imaging agent in said sample from an unaffected subject or from an unaffected area of the test subject, is an indication that the test subject has a cancer.

In one embodiment, the cancer is selected from the group consisting of head and neck cancer, liver cancer, pancreatic cancer, esophageal cancer, stomach cancer, lung cancer, breast cancer, ovarian cancer, uterine cancer, endometrial cancer, cervical cancer, prostate cancer, adrenal cancer, lymphoma, salivary gland cancer, bone cancer, brain cancer, cerebellar cancer, colon cancer, rectal cancer, colorectal cancer, oronasopharyngeal cancer, NPC, kidney cancer, bladder cancer, skin cancer, melanoma, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, and adenocarcinoma.

In one embodiment, the cancer is a metastatic cancer.

Optionally, a therapeutic agent can be attached or can be included in a pharmaceutical composition comprising the imaging complex.

In one aspect, the imaging agent is detected with a PET or SPECT/CT scanner coupled to a computer, and analyzing imaging data using a program. In one aspect, the method detects the location of the cancer in the subject. In one aspect, the invention provides for detecting and imaging a cancer which has metastasized, i.e., the method can detect cancer in multiple locations in the same subject.

The invention is also useful for determining the stage of carcinogenesis of a cancer and monitoring its progression from early to late stage cancer. This method is useful for determining the type and amount of therapy to use.

Optionally, a therapeutic agent can be attached to or can be included in a pharmaceutical composition comprising the imaging complex.

Clinically relevant PET/SPECT tracers as used herein enable the detection of small tumors and metastases.

In one aspect, the imaging agent or detectable moiety includes, but is not limited to, a radionuclide, a radiological contrast agent, a paramagnetic ion, a metal, a biological tag, a fluorescent label, a chemiluminescent label, an ultrasound contrast agent and a photoactive agent.

Therapeutic Agents

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes, or other agents may be used as adjunct therapies when using the liposome complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents, and combinations thereof. In one aspect, the drug or agent is encapsulated into a liposome of the invention.

One of ordinary skill in the art will appreciate that various kinds of molecules and compounds can be delivered to a cell or tissue using the mannosylated-liposomes of the invention. Liposomes have been used as carriers for delivering many types of molecules.

Imaging and Diagnostic Agents

Diagnostic agents are selected from, for example, the group consisting of a radionuclide, a radiological contrast agent, a paramagnetic ion, a metal, a fluorescent label, a chemiluminescent label, an ultrasound contrast agent, and a photoactive agent. Such diagnostic agents are well known and any such known diagnostic agent may be used. Non-limiting examples of diagnostic agents may include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn ⁵⁵Co, ⁷²As, ⁷⁵Br, ⁷⁶Br, ⁸²mRb, ⁸³Sr, or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas-filled liposomes.

### Positron Emission Tomography (PET)—

Positron emission tomography is a nuclear medicine imaging technique which produces a three-dimensional image or picture of functional processes in the body. The theory behind PET is simple enough. First a molecule is tagged with a positron-emitting isotope. These positrons annihilate with nearby electrons, emitting two 511 keV photons, directed 180 degrees apart in opposite directions. These photons are then detected by the scanner which can estimate the density of positron annihilations in a specific area. When enough interactions and annihilations have occurred, the density of the original molecule may be measured in that area. Typical isotopes include ¹¹C, ¹³N, ¹⁵O, ¹⁸F, ⁶⁴Cu, ⁶²Cu, ¹²⁴I, ⁷⁶Br, ⁸²Rb and ⁶⁸Ga, with ¹⁸F being the most clinically utilized. Although some PET probes must be made with a cyclotron and have a half-life measured in hours, forcing the cyclotron to be on site, PET imaging does have many advantages though. First and foremost is its sensitivity: a typical PET scanner can detect between 10⁻¹¹ mol/L to 10⁻¹² mol/L concentrations.

Some agents used for PET imaging provide information about tissue metabolism or some other specific molecular activity. Following are commonly used agents or potential agents for use with the compositions and methods of the invention or in combination with the other compositions and methods of the invention:

⁶⁴Cu-ATSM: ⁶⁴Cu diacetyl-bis(N⁴-methylthiosemicarbazone), also called ATSM or Copper 64, is an imaging agent used in PET or PET/CT for its ability to identify hypoxic tissue (tissue with low oxygen).

FDG: ¹⁸F-fluorodeoxyglucose (FDG) is a radioactive sugar molecule, that, when used with PET imaging, produces images that show the metabolic activity of tissues. In FDG-PET scanning, the high consumption of the sugar by tumor cells, as compared to the lower consumption by normal surrounding tissues, identifies these cells as cancer cells. FDG is also used to study tumor response to treatment.

¹⁸F-fluoride: ¹⁸F-fluoride is an imaging agent for PET imaging of new bone formation. It can assess changes both in normal bone as well as bone tumors. As a result, it can be used to measure response to treatment.

FLT: 3′-deoxy-3′-[¹⁸F]fluorothymidine (FLT) is a radiolabeled imaging agent that is being investigated in PET imaging for its ability to detect growth in a primary tumor. Studies may also measure the ability of FLT with PET to detect tumor response to treatment.

FMISO: ¹⁸F-fluoromisonidazole is an imaging agent used with PET imaging that can identify hypoxia (low oxygen) in tissues. Tumors with low oxygen have been shown to be resistant to radiation and chemotherapy.

Gallium: Gallium attaches to areas of inflammation, such as infection. It also attaches to areas of rapid cell division, such as cancer cells. It can take gallium a few days to accumulate in the affected tissue, so the scan may be done 2-3 days after the gallium is administered.

Technetium-99m: Technetium-99m is used to radiolabel many different common radiopharmaceuticals. It is used most often in bone and heart scans.

Thallium: Thallium is a radioactive tracer typically used to examine heart blood flow. The thallium scan is often combined with an exercise test to determine how well the heart functions under stress. A thallium scan may also be used to measure tumor response.

Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerythrin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Techniques for detecting and measuring these agents are provided in the art or described herein.

Detecting the location of the imaging agent may be conducted by any suitable technique known to one skilled in the art, for example, by positron emission tomography (PET).

Yet another modification may comprise the introduction of one or more detectable labels or other signal-generating groups or moieties, depending on the intended use of the labeled molecule. Suitable labels and techniques for attaching, using and detecting them will be understood by one of ordinary skill in the art, and for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be understood by the skilled artisan, and for example, include moieties that can be detected using NMR or ESR spectroscopy. Such labeled molecules of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link a molecule of the invention to a protein, polypeptide or chemical compound that is bound to the other half of the binding pair, i.e., through formation of the binding pair. For example, such a conjugated molecule may be used as a reporter, for example, in a diagnostic system where a detectable signal-producing agent is conjugated to avidin or streptavidin. One non-limiting example are the liposomal formulations described by Cao and Suresh, Journal of Drug Targeting, 8, 4, 257 (2000). Such binding pairs may also be used to link a therapeutically active agent to a molecule of the invention for use in or attached to a liposome of the invention.

A number of trivalent metal radionuclides have physical properties suitable for radioisotope imaging (e.g., indium-111 (¹¹¹In) gallium-67/68 (^{67/68}Ga) and yttrium-86 (⁸⁶Y)) or for targeted radionuclide therapy (e.g., ⁹⁰Y and lutetium-177 (¹⁷⁷Lu)). These metal radionuclides can be combined with a targeting biomolecule or entity (such as a peptide or antibody or mannosylated liposome) in order to diagnose, monitor or treat disease. To obtain a radiolabeled molecule or entity with the required stability, the peptide or protein or mannosylated liposome must first be conjugated to a suitable chelator, or have it incorporated into it, in order to complex the metal. The complexes should be stable in biological systems. Most often, diethylenetriaminepentaacetic acid (DTPA) and/or 1,4,7,10-tetraazacyclododecane-1,4,7,10-tetraacetic acid (DOTA; CAS 60239-18-1) are used (see Choe and Lee, 2007, Current Pharmaceutical Design, 13:17-31; Li et al., 2007, J. Nuclear Medicine, “⁶⁴Cu-Labeled Tetrameric and Octameric RGD Peptides for Small-Animal PET of Tumor avb3 Integrin Expression”, 48:1162-1171; Nahrendorf et al, 2009, JACC Cardiovasc. Imaging, 2:10:1213-1222; Li et al., 2009, Mol. Cancer Ther., 8:5:1239-1249; Yim et al., 2010, J. Med. Chem., 53:3944-3953; Dijkgraaf et al., 2010, Eur. J. Nucl. Med. Mol. Imaging, published online 21 Sep. 2010; U.S. patent application Ser. No. 10/792,582; Dransfield et al., U.S. Pat. Pub. Nos. US 2010/0261875; U.S. Pat. No. 7,666,979). Of the metals mentioned, the DOTA complexes are more thermodynamically and kinetically stable than the DTPA complexes (see Sosabowski et al., Nature Protocols 1, -972-976 (2006) and Leon-Rodriguez et al., Bioconjugate chemistry, Jan. 3, 2008; 19(2):391-402).

### Chelating Agents

In some embodiments, a chelating agent may be attached to or incorporated into a liposome of the invention, and used to chelate a therapeutic or diagnostic agent, such as a radionuclide. Exemplary chelators include but are not limited to DTPA (such as Mx-DTPA), DOTA, TETA, NETA or NOTA. Methods of conjugation and use of chelating agents to attach metals or other ligands to proteins are well known in the art (see, e.g., U.S. patent application Ser. No. 12/112,289, incorporated herein by reference in its entirety).

Useful chelators encompassed by the invention include, but are not limited to, DTPA, DO3A, DOTA, EDTA, TETA, EHPG, HBED, NOTA, DOTMA, TETMA, PDTA, TTHA, LICAM, HYNIC, and MECAM. HYNIC is particularly useful for chelating Tc99, another imaging agent of the invention.

### EXAMPLES

### Materials and Methods

Animal Model.

All experiments were carried out under protocols approved by the Institutional Animal Care and Use Committee. Our mouse model is based on one reported by Blackwell's group. Female FVB mice (Jackson Laboratory, n=6) aged 6-8 weeks received weekly intraperitoneal (IP) injections of 1 mg urethane/g body weight dissolved in sterile 0.9% NaCl. Control mice (n=3) received saline IP injections. Twenty weeks after the initial urethane injection, MRI was used to verify lung tumor presence. PET imaging was performed when at least one lung tumor reached 1.5 mm in diameter.

Prior to in vivo studies, we characterized macrophage infiltration at the tumor border in the urethane-FVB mouse model. Twenty-four weeks after urethane treatment, lungs from a representative mouse were harvested en bloc and inflated with formalin through the trachea. After formalin fixation, the lungs were embedded in paraffin, cut into 2 μm slices, and hematoxylin and eosin (H&E)-stained. Slides were digitized and examined for tumors.

To evaluate TAM density relative to macrophage density in normal lung, we immunostained lung sections (n=4 urethane-treated mice) with rat anti-mouse F4/80 monoclonal antibody (AbDSerotec). F4/80 is a well-known, highly specific macrophage molecule in mice. To estimate macrophage density, regions of interest (ROIs) were drawn in areas corresponding to stroma and normal lung. Macrophages were counted in areas of known size and macrophage density (MØ per square millimeter) was calculated. Ten ROIs were drawn in each compartment and the average density was computed.

Finally, we used confocal microscopy to estimate the percentage of F4/80⁻ positive cells that overexpress CD206. Lungs (n=2 mice, 24 weeks post-urethane) were harvested and inflated with 1.5 mL of Optimum Cutting Temperature (OCT; Fisher Scientific) through the trachea. The lungs were snap-frozen in liquid nitrogen, frozen into a block, and cryosectioned coronally. Frozen lung tissue sections were fixed and incubated with rat anti-mouse F4/80 and rabbit anti-mouse CD206 (AbDSerotec). After primary antibody incubation, sections were treated with Alexa Fluor 594-conjugated goat anti-rat IgG and Alexa Fluor 488-conjugated goat anti-rabbit IgG. Cell nuclei were visualized by 4′,6-diamidino-2-phenylindole (DAPI). Confocal images of tumor regions were captured using a fluorescence microscope (Carl Zeiss LSM 700). To estimate the percentage of cells within the tumor stroma that co-express F4/80 and CD206, thresholding was applied to several images spanning different tumor regions across two mice.

Liposome Preparation and Characterization.

Liposomes were composed of 18.8 mg/mL of L-α-Phosphatidylcholine, 4.2 mg/mL of cholesterol, and 0.025 mg/mL of the lipophilic fluorescent probe 3,3′-Dioctadecyloxacarbocyanine Perchlorate (DiO, λₑₓ=484 nm, λₑₘ=501 nm, Molecular Probes, USA). The liposomes were made using dehydration-rehydration: the lipids and DiO were dissolved in chloroform, the solvent was evaporated, and the resultant thin-film hydrated with a 10 mM solution of chelating agent 1,4,7,10-tetra-azacyclododecane-1,4,7,10-tetraacetic acid (DOTA) in 10 mM 4-(2-Hydroxyethyl)-1-piperazine-Ethanesulfonic Acid (HEPES) buffer with 150 mM NaCl and a pH of 4 for 2 hours at 37° C. and overnight at 4° C. The liposome solution was freeze-thawed 5 times and then extruded consecutively 20 times through 1 μm, 600 nm, 400 nm and 200 nm polycarbonate membrane filters using a Lipex extruder with high-pressure nitrogen. The non-encapsulated DOTA was removed by dialysis using a Slide-A-Lyzer G2 dialysis cassette with a molecular weight cut-off of 10,000 against five-2 liters of HEPES buffer containing 150 mM NaCl (pH 7.4).

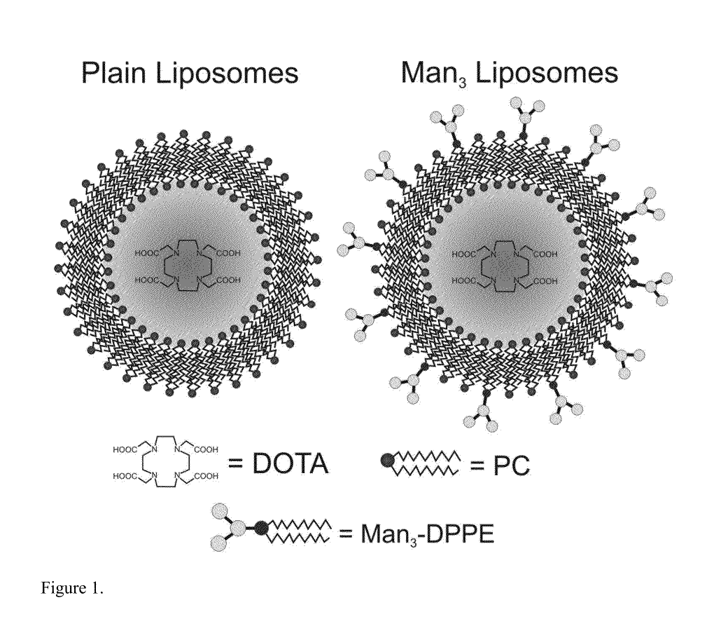

Mannosylated encapsulated liposomes (MAN-LIPs) were prepared as described above with the inclusion of neoglycolipids synthesized from mannotriose and dipalmitoylphosphatidylethanolamine (DPPE) by reductive amination (manufactured to our specification by Encapsula NanoSciences, Nashville, Tenn.). The mannosylated phospholipid was added at a 1:20 MAN-DPPE to phosphatidylcholine (PC) molar ratio and dissolved in chloroform. The mean particle diameter was verified by a laser light scattering particle size analyzer (LS-900, Otsuka Electronics). A schematic diagram of plain and MAN-LIPs is in FIG. 1.

⁶⁴Cu Labeling.

Remote loading was used to radiolabel DOTA-containing liposomes with the PET probe ⁶⁴Cu (t_{1/2}=12.7 h), by utilizing the lipophilic transporter hydroxyquinoline to ferry ⁶⁴Cu to the liposome interior where it is more tightly chelated by the encapsulated DOTA. Copper loading of the liposomes was confirmed using size exclusion chromatography (SEC) column to determine if the fluorescent dye DiO labeled liposomes eluted in the same fractions as the radioactive ⁶⁴Cu.

Culture of Murine BMDM.

To create primary macrophage cultures that mimic physiological and surface marker characteristics of macrophage phenotypes observed in vivo, bone marrow (BM) was flushed from the femurs and tibiae of FVB mice 8-12 weeks of age. The cell suspension was filtered using a 70-mm filter mesh, centrifuged at 500×g, and re-suspended in 1.0 mL of MACS buffer (phosphate buffered saline (PBS) supplemented with 1% bovine serum albumin (BSA) and 2 mM ethylenediaminetetraacetic acid). Four 10 cm polystyrene Petri plates were prepared with 8 mL complete medium: α-Minimum Essential Medium (α-MEM; Gibco) supplemented with 10% heat-inactivated fetal bovine serum, 10% CMG14-12 cell conditioned medium (as a source of macrophage-colony stimulating factor (M-CSF)), and 1% pen/strep (complete α-MEM). The cell suspension was pipetted onto the Petri dishes and cultured 4 days at 37° C. with 5% CO₂ in a humidified chamber. This resulted in cells that were 95% positive for F4/80, consistent with other reported results. After the 4 day incubation, adherent cells were treated for 10 min at 37° C. with 1.0 mL of trypsin (Gibco) diluted 1:10 in PBS. Recovered cells were counted using a hemacytometer and re-plated in 60 mm Petri dishes with 3 mL complete α-MEM at a density of 1.0×10⁶ cells per well. 24 hours after re-plating, the dishes were divided and treated for 24 hours with: mouse recombinant interferon-γ (IFN-γ; PeproTech, Rocky Hill, N.J., 20 ng/mL) and lipopolysaccharide (LPS; Sigma-Aldrich, 100 ng/mL) resulting in M1-like macrophages (M1-MØ), and; mouse recombinant interleukin-4 (IL-4; PeproTech, Rocky Hill, N.J.) plus IL-13 (PeproTech) at a concentration of 20 ng/mL each, resulting in M2-like macrophages (M2-MØ). In vitro stimulation of murine BM-derived macrophages with IL-4 and IL-13 has been shown to induce M2a or wound healing macrophages similar to TAMs. Therefore, we used Western blot analysis and flow cytometric analysis to assess mannose receptor expression to verify M2 polarization after cytokine treatment.

Evaluation of Liposome Uptake by MØ In Vitro.

Uptake of liposomes by macrophages was evaluated by flow cytometry (FACSCalibur, BD Biosciences). M2-MØ and M1-MØ were incubated with either MAN or plain liposomes at a concentration of 960 nmol phospholipid per mL of PBS at 37° C. for 90 min. Cells that were incubated with PBS served as negative controls. Following incubation, the cells were washed three times in MACS buffer to remove non-associated liposomes and detached from the Petri dish with No-Zyme solution (Sigma). To measure cell surface mannose receptor expression, cells were incubated with anti-Fc receptor mAb for 60 minutes on ice to block non-specific binding of antibody to the mouse Fcγ domain, then were incubated for 20 min with rat anti-mouse CD206 directly conjugated to Alexa Fluor 647 (AbD Serotec). The cells were then fixed with Cytofix (BD Biosciences) and transferred to flow tubes. Two-color flow cytometry was performed with 10,000 events acquired per sample. The fluorescent amplifiers of the FL-1 and FL-4 detector filters were adjusted to ensure that the negative cell population appeared in the first logarithmic decade. Compensation for spectral overlap was not required due to sufficient separation between the fluorescent emission profiles. All experiments were done in triplicate.

FlowJo (TreeStar Inc.) was used to analyze the raw flow data. Macrophages were identified by their light scattering properties. Using negative controls, cellular autofluorescence in both channels was determined by manually defining a gate that maximally included 1% of autofluorescent cells. Only cells exhibiting a fluorescence intensity above this gate were included in the analysis. CD206 expression was quantified in each treatment group as the geometric mean fluorescence intensity (MFI) of positive cells. Similarly, the geometric MFI of DiO fluorescence associated with M2-MØ and M1-MØ following liposome incubation was measured.

In Vivo Studies of Liposome Distribution and Uptake by MØ.

To determine the blood half-life of radiolabeled MAN and plain liposomes, repetitive tail-vein bleeds (about 50 μL of blood per sample) were performed at 5 and 30 min, and 1.5, 3, 6, and 18 h after intravenous (IV) injection in 6 normal FVB mice. Liposome dose was 1.9 μmol total phospholipid labeled with 50-75 μCi (1.85-2.8 MBq) of ⁶⁴Cu in a total volume of 160 μL. The radioactivity of each sample was measured using a γ-counter calibrated for ⁶⁴Cu energy. In order to estimate how much radioactivity was in the total blood volume at each time point, we used a value of 7.3% of the body mass for total blood mass. From this we calculated the percentage of the injected dose in total blood mass (% ID_{TBM}) at each time point using:

$$\% {ID}_{TBM} = \frac{( \frac{μ {Ci}_{sample}}{{g}_{sample}} ) \times BW \times 0.073}{μ {Ci}_{ID}} \qquad Eq . 1$$

where μCiₛₐₘₚₗₑ, gₛₐₘₚₗₑ, μCi_{ID}, and BW represents the radioactivity measured in the blood sample, the mass of the blood sample, the radioactivity of the injected dose (ID), and the body weight of the mouse, respectively.

Twenty-four (24) weeks after urethane treatment, lung tumor bearing mice were IV injected with ⁶⁴Cu-labeled MAN-LIPs via the lateral tail vein. Each mouse received 1.9 μmol total phospholipid labeled with 400-500 μCi (14.8-18.5 MBq) of ⁶⁴Cu for a total volume of 160 μL. Mice were imaged using a Focus 120 PET scanner (Siemens, Knoxyille, Tenn.). During the 40 min PET acquisition, anesthesia was maintained using 1.25% isoflurane in O₂ inhaled through a nose cone. Heart rate, respiration, and rectal temperature were monitored (SAII, Stony Brook, N.Y.). PET data were reconstructed using OSEM algorithm with 2 iterations and 12 subsets followed by MAP algorithm (18 iterations). The reconstructed image (not corrected for attenuation) was composed of 95 axial slices of thickness 0.79 mm with an in-plane voxel dimension of 0.4 mm×0.4 mm (128×128 pixels). To determine the optimal time point after IV liposome injection for imaging, a preliminary study was conducted in which tumor to normal lung uptake ratios were computed on serial scans acquired over 18 h in 4 mice.

Immediately after the PET scan, MR images were acquired on a 4.7 T MRI system (Varian, Inc., Palo Alto, Calif.) to identify lung and tumor boundaries on the PET scans. MRI used a cardiac and respiratory gated multi-slice, spin-echo sequence developed in our lab with the following parameters: field of view=25.6 mm, effective matrix=128×128, slice thickness=0.6 mm, TR=168 ms, TE=11 ms, number of averages=4, number of slices=10, and number of interleaves=4, Gadolinium-DTPA (Magnevist; Bayer Schering Pharma, Berlin, Germany) 50 μmol/kg BW at a dose of 50 μmol/kg body weight. The total scan time was 6-8 minutes per interleave, depending on the heart rate and breathing rate. Four 10-slice interleaved stacks were acquired to cover the entire lung field. To facilitate PET-MRI co-registration, a custom-designed multi-modality fusion phantom was scanned to determine the rigid-body transformation matrix for fusing subsequent mouse data sets.

Using AMIDE software, MAN-LIP uptake in tumor and remote lung tissue, liver, and muscle was quantified with PET using co-registered, resolution-matched MR images to guide the size and location of PET ROIs. AMIDE: “A Medical Imaging Data Examiner” is a tool for viewing, analyzing, and registering volumetric medical imaging data sets. it was developed using GTK+/GNOME, and runs on any system that supports the toolkit (Linux, Mac OS X with fink, etc.). For lung tumors, ROIs were carefully drawn around the tumor perimeter for each slice in which the tumor was visible. The percent injected activity per μL (% ID/μL) was computed for each tissue type by dividing the ROI-derived tissue tracer concentration by the radioactivity of the injected dose. Due to their small size, ROI-derived radioactivity concentration measurements in lung tumors suffer from partial volume effects (PVE). We corrected for PVE by multiplying the ROI-derived uptake concentration measured in the tumor by a recovery coefficient (RC) that was obtained using a hot-rod phantom containing a known radioactivity concentration. The phantom images allowed us to construct a look-up table that allows an RC value to be estimated based on the diameter of the object (tumor). Using MRI, we measured the average diameter for each lung tumor and assigned to it an RC value that was used to correct the ROI-derived radioactivity concentration.

Ex Vivo Tissue Imaging.

Immediately following imaging, the animals were euthanized and the lungs, liver, and spleen were harvested. The lungs were inflated with 1.5 mL of formalin through the trachea, which was then tied off with a suture. Tissues were washed and rescanned by PET with the same parameters and acquisition time described above. The primary motivation for acquiring ex vivo PET scans was to image the lungs without the presence of signal contamination originating from the liver. Fluorescence imaging of the harvested organs was also performed using the IVIS Spectrum to examine the tissue distribution of DiO and its correlation with ⁶⁴Cu. For DiO fluorescence imaging, we used an excitation filter centered at 500 nm and an emission filter centered at 540 nm. The lamp level was set to high, binning to medium, field of view to 6.4-12.3 cm depending on object size, f number to 2, and exposure time to 0.5-1 s such that no saturation occurred in the image.

Two representative mice that were administered MAN-LIPs were chosen for further analysis by confocal fluorescence microscopy. For this study, lungs were harvested after in vivo imaging and inflated with 1.5 mL of OCT via the trachea and snap frozen in liquid nitrogen. The lung tissue was then mounted in OCT embedding compound and stored at −80° C. until cut into 4 μm slices. Tumor-positive frozen tissue sections were fixed and incubated with rat anti-mouse F4/80 followed by Alexa Fluor 594-conjugated goat anti-rat IgG. For visualization of cell nuclei, slides were counterstained with DAPI. Each fluorophore was carefully selected to minimize spectral overlap and potential bleed-through artifacts. Immunofluorescent staining of frozen lung tissue was done by IHC Tech (Boulder, Colo.). All slides were analyzed using a Zeiss LSM 700 confocal scanning microscope.

Co-Injection Study.

Even though the incorporation of mannotriose into liposome formulations has been shown to enhance macrophage uptake over plain liposomes both in vitro and in vivo, we set out to determine the significance of liposome mannosylation for targeting TAMs in vivo in our urethane animal model. The first study, intended to address the importance of liposome mannosylation in mediating uptake by TAMs, involved the co-injection of MAN and plain liposomes (FIG. 1) into lung tumor-bearing mice. The second study was identical to the first, with the exception that the plain liposomes were prepared with the inclusion of polyethyleneglycol (PEG)-bearing lipids at a molar ratio of 1:20 with respect to PC. The intent of this study is to find out what property is more critical for TAM targeting in vivo by IV injection: the inclusion of PEG which promotes long blood circulation time and retention at the tumor site due to the Enhanced Permeability and Retention effect, or the inclusion of mannose which may mediate enhanced liposome recognition and endocytosis. In order to independently detect each liposome type based on fluorescence, MAN-LIPs were prepared and labeled with DiO. However, plain liposomes (with and without PEG) were labeled with a red-shifted lipophilic dye (1,1′-Dioctadecyl-3,3,3′,3′-Tetramethylindodicarbocyanine (DiD), λₑₓ=648 nm, λₑₘ=670 nm, Molecular Probes, USA) whose excitation/emission profile did not overlap with DiO.

For these in vivo studies, each mouse was tail-vein injected with 1.9 μmol total phospholipid (approximately 80 μL) of each liposome type plus 80 μL of HEPES buffer for a total injected volume of 240 μL. Six hours after injection, the mice were euthanized and their lungs, liver, and spleen were harvested. Lungs were inflated with 1.5 mL of OCT via the trachea and carefully arranged on black construction paper along with the liver and spleen. Fluorescent images were acquired using the IVIS Spectrum system. Radiant efficiency for DiO and DiD, defined as the emission light detected (photons/sec/cm²/str) normalized by the illumination power density (μW/cm²) was measured using the Living Image 4.0 (Xenogen Corp., Alameda, Calif.). Tumor-to-remote lung, tumor-to-liver, and tumor-to-spleen ratios were computed in mice co-injected with MAN-LIPs and plain liposomes (n=2) and MAN-LIPs and PEG liposomes (n=2).

In addition to whole lung ex vivo fluorescence imaging, we also investigated liposome uptake by TAMs at the cellular level. Immediately after imaging, lungs were snap frozen and stored at −80° C. until cut into 4 μm slices. Select tumor-positive tissue sections were further stained with DAPI and rat anti-mouse F4/80 followed by TRITC-conjugated goat anti-rat IgG. Confocal microscopy was used to examine the relative presence of MAN versus plain liposomes and MAN versus plain PEG liposomes within TAMs.

Statistical Analysis.

For comparison of the expression of CD206 and liposome association by macrophage treatment groups in vitro, a two-tailed unpaired Student's t-test was used for evaluating statistical significance. All data shown are representative of at least 3 independent experiments. The same statistical test was also used to compare tumor to remote lung liposome uptake measured from PET images and macrophage densities in different tissue compartments measured from immunohistochemistry images. P values were considered to be statistically significant when less than 0.05.

### Results

Validation of Urethane Model.

Twenty-four weeks after urethane treatment, lung tumors were clearly visible on H&E-stained tissue. The majority of tumors were located on the lung pleural surface, an observation consistent with other studies. Quantification of F4/80-positive cells on immunohistochemistry images revealed that tumor stroma had a seven-fold higher macrophage density compared to remote lung (673±196 vs. 89±29 MØ/mm²). It is interesting to note that there was no statistically significant difference between remote lung macrophage density in tumor-bearing mice (89±29 MØ/mm²) and macrophage density in lung tissue of saline-treated mice (85±29 MØ/mm²). This result suggests that lung tumors do not influence macrophage density in remote lung compared to that of normal lung in aged-matched, saline-treated mice.

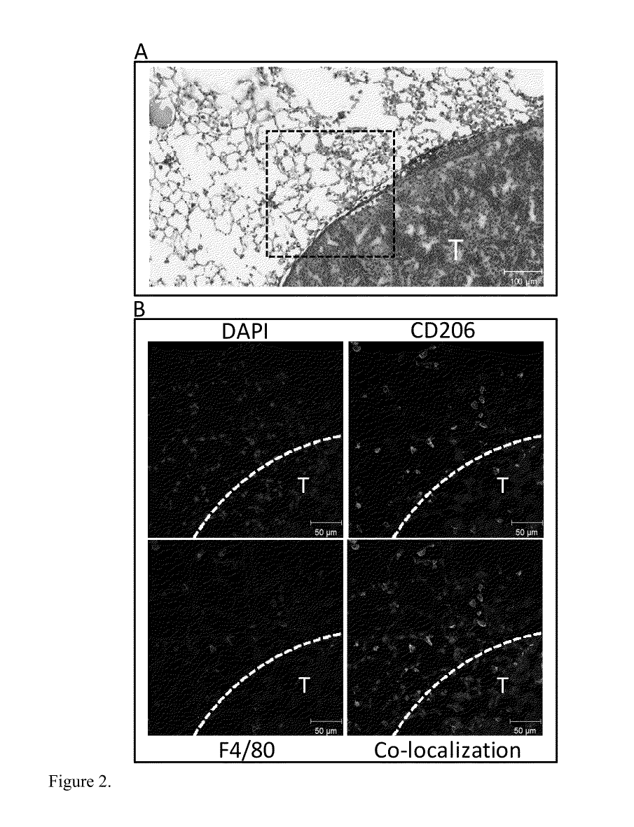

FIG. 2 shows two contiguous lung sections stained in two different ways. The first section (FIG. 2A) was H&E stained, which allowed clear identification of tumor and immune cell infiltration in the surrounding stroma. The second section underwent double-immunofluorescence staining to allow simultaneous visualization of F4/80 and CD206 expression in H&E-confirmed tumor areas. Individual color channels of the confocal image, spatially corresponding to the area in the H&E-stained image indicated by the black dashed box, are shown in FIG. 2B. Consistent with immunohistochemical analysis, there was moderate accumulation of F4/80+ macrophages at the tumor border and significantly fewer macrophages within the tumor. At the border of the tumor (indicated by the dashed white line), the majority of F4/80-positive macrophages (red) also stain positive for CD206 (green), signifying an M2 macrophage phenotype consistent with other reported studies based on a similar urethane model.

To estimate the percentage of F4/80-positive cells that co-express CD206, we used a thresholding technique based on staining intensity. For both the red (F4/80) and the green (CD206) channels, cells were identified and the percent of cells that were positive for both F4/80-positive and CD206-positive cells was computed. After pooling together the results from all images, we estimate that 94% of F4/80′ also stain for CD206, indicating that a great majority of macrophages in the tumor stroma are M2-like.

Liposome Characterization.

Successful conjugation of MAN with DPPE (MAN-DPPE) was confirmed by MALDI-TOF mass spectrometry. The m/z spectrum demonstrated a strong peak of 1180.6 for the protonated molecule consistent with the theoretical mass value of 1180.7. As determined by laser light scattering, liposomes exhibited a Gaussian size distribution with the peak of the Gaussian curve aligning with the pore size of the membrane (200 nm). The majority of the extruded population was between 180 and 220 nm.

The remote labeling of liposomes by the PET radionuclide ⁶⁴Cu was verified by SEC. The chromatography profile revealed that the peak of ⁶⁴Cu elution aligned with the peak of fluorescence (DiO) elution, confirming that ⁶⁴Cu was entrapped in the liposomes upon exiting the column. The elution of free ⁶⁴Cu alone occurred much later when passed through the column separately. Nearly 100% of the starting radioactivity was recovered in the void volume and was associated with the liposome fractions.

In Vivo Liposome Uptake.

We measured mannose receptor expression in the cultured macrophages. CD206 expression was upregulated on M2-MØ compared to M1-MØ as measured by flow cytometry and Western blotting, respectively. The uptake of plain and MAN-LIPs by M1-MØ and M2-MØ was assessed by flow cytometry.

Following identification based on scatter properties, macrophages were analyzed for DiO fluorescence. Fluorescence histograms of M1-MØ and M2-MØ following 90 min incubation with either DiO-labeled plain or MAN liposomes showed that MAN-LIPs were more strongly associated with macrophages of both stimulation groups compared to plain liposomes. However, quantification of the flow cytometry data revealed a significant 1.9-fold higher association of MAN-LIPs with M2-MØ compared to M1-MØ (P<0.05). This enhanced association is likely due to receptor recognition of liposome mannotriose and subsequent mannose receptor-mediated endocytosis.

In Vivo Studies.

The blood clearance of ⁶⁴Cu labeled liposomes was studied in normal healthy FVB mice after administration of 10 MBq of radiolabeled liposomes through the lateral tail vein. The blood clearance of both liposomes followed monoexponential decay with half-lives of 0.33 and 0.23 h for MAN-LIPs and plain liposomes, respectively. This result is consistent with previously published work which measured a blood half-life of less than 30 min for liposomes of similar size and composition (PC and cholesterol).

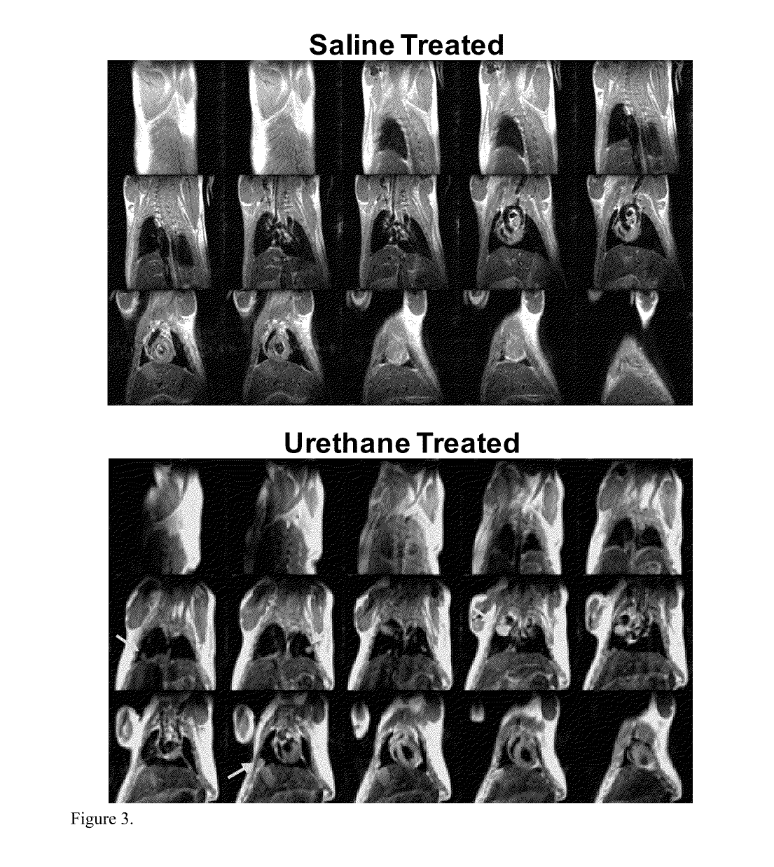

Examples of coronally acquired spin-echo MRI of a urethane and saline-treated mouse are shown in FIG. 3. Several tumors are clearly visible in the lungs of the urethane-treated mouse, which are easily detected relative to normal lung tissue which is nearly void of signal. In contrast, the saline-treated mouse has no detectable lung tumors.

To assess in vivo TAM targeting, ⁶⁴Cu-labeled MAN-LIPs were IV injected into lung tumor-confirmed mice (n=6). PET imaging was performed 6 h following injection. This time point was chosen because it resulted in the highest ⁶⁴Cu signal ratio between tumor tissue and remote lung while still maintaining sufficiently high radioactivity counts. Furthermore, after 6 h approximately 1% of the injected dose remains in the blood circulation, minimizing potential signal contamination from ⁶⁴Cu-MAN-LIPs that may reside in the vasculature.

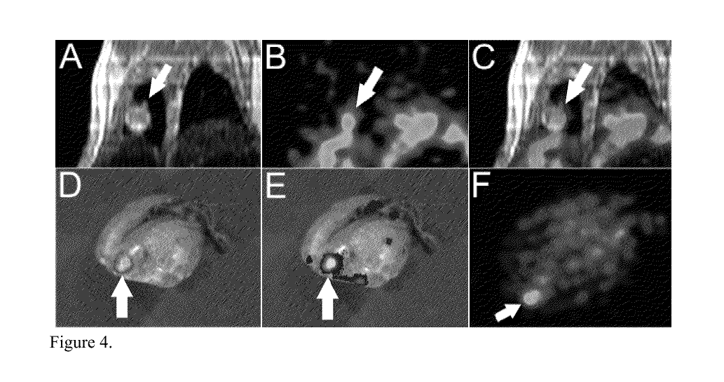

FIG. 4A shows a coronal MRI revealing a tumor in the right lung (white arrow) and a PET image (FIG. 4B) showing the distribution of MAN-LIPs 6 h post injection. Fused data sets (FIG. 4C) confirm high tumor localization of the PET signal compared to normal lung tissue. Following the in vivo scans, the lungs were excised and imaged by PET without the spillover from the nearby liver. The distribution of DiO-labeled liposomes within the excised lung was evaluated by fluorescence imaging acquired using the IVIS Spectrum. This information could not be obtained in vivo due to limited tissue penetration and photon scattering. A photo of the excised lung clearly shows the tumor (arrow, FIG. 4D) as well as several other smaller tumors. The fluorescence image overlaid on the photo reveals a strong DiO-fluorescent signal within the tumor and minimal accumulation in non-tumor areas of the lung (FIG. 4E). Ex vivo PET maximum intensity projection image, shown in FIG. 4F, shows focal ⁶⁴Cu signal in the area of the lung spatially corresponding to the tumor and the fluorescence signal as shown in FIG. 4D-E.

The average diameter of lung tumors measured across all mice studied ranged from 1.25 mm to 2.65 mm. This corresponded to RC values of 0.32 to 0.72. The PV-corrected uptake of ⁶⁴Cu-labeled MAN-LIPs in lung tumors was 9.62±2.49% ID/μL at 6 h post injection. In contrast, uptake in normal lung tissue was significantly lower (1.4±0.6% ID/μL) resulting in a tumor-to-normal lung ratio of 6.7.

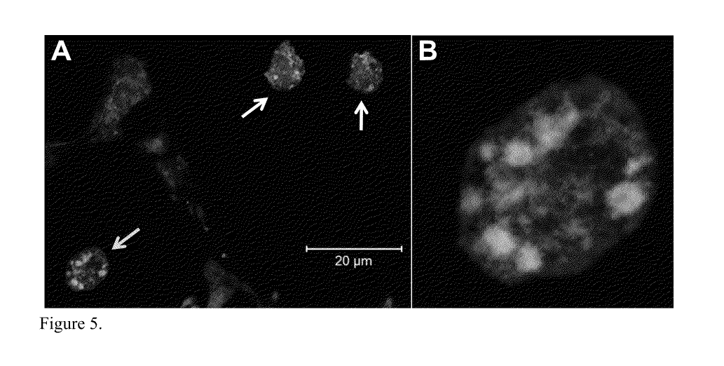

Following PET imaging, the dual-labeled MAN-liposome permitted a detailed assessment of cellular distribution by confocal fluorescence microscopy within lung tumor tissue. As shown in FIG. 5A, significant DiO fluorescence was associated with F4/80-positive cells (TAMs). (In this image, the tumor was not captured in the field of view but was located immediately to the right). Furthermore, this fluorescence was not limited to the circumference of TAMs, but rather was clustered within them as clearly seen on the enlarged view of the TAM (FIG. 5B) indicated by the yellow arrow. This clustered distribution of DiO fluorescence is consistent with liposome entrapment in endosomal structures, signifying that tumor enhanced in vivo PET signals were the result of MAN-liposome-labeled TAMs.

Co-Injection Results.

In order to accurately assess liposome lung distribution following co-injection, we first demonstrated that the fluorescence associated with each liposome type could be separately detected. Using our equipment, the fluorescent dye DiO could be excited and detected apart from diD using an excitation and emission filter pair of 500 nm and 540 nm, respectively. Conversely, DiD could be detected apart from DiO using an excitation and emission filter pair of 605 nm and 640 nm, respectively. This was expected based on their non-overlapping excitation and emission spectra.

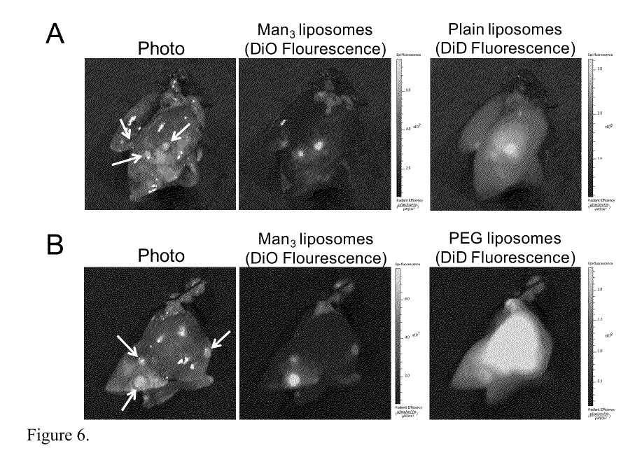

Representative ex vivo fluorescence images of lungs 6 h after the co-injection of MAN and plain liposomes as well as MAN and PEG liposomes are shown in FIG. 6A, and FIG. 6B, respectively. From these images it was clearly evident that MAN and plain liposomes strongly localized at lung tumors, however, MAN liposomes exhibited a lower background signal in non-tumor lung tissue. PEG liposomes showed a diffuse lung distribution and consequently poor tumor contrast likely due to their enhanced blood circulation time.

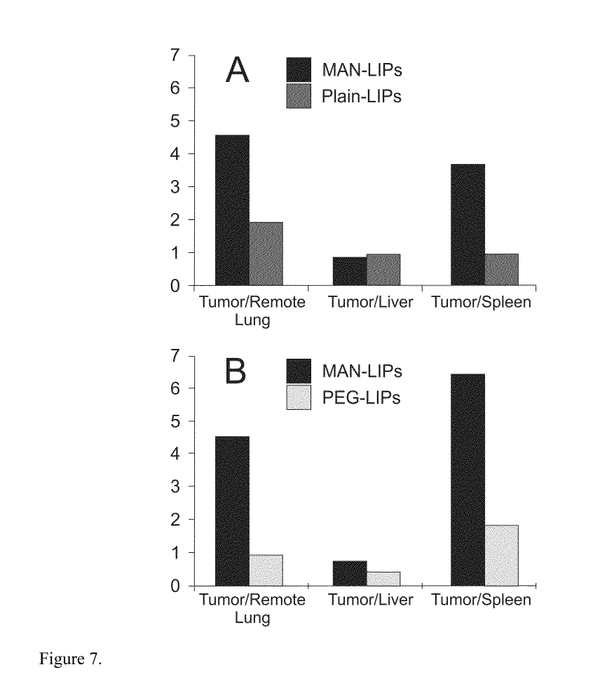

To examine uptake in various tissues, we computed uptake ratios between the tumor and remote lung, tumor and liver, and tumor and spleen for each liposome type based on the fluorescence images. Tissue ratios were computed to avoid potential biases in the excitation of the fluorescent dyes as well as to avoid biases due to differences in signal attenuation in animal tissues. As shown in FIG. 7A, MAN-LIPs exhibited a higher tumor-to-remote lung (4.6) and tumor-to-spleen (3.7) ratio compared to plain liposomes. However, the tumor-to-liver ratio was comparable (0.8). MAN-LIPs also exhibited a high tumor-to-remote lung ratio (1.4) when co-injected with PEG liposomes. PEG liposomes exhibited the lowest tumor-to-remote lung ratio (0.9) of the three liposomes studied. This is likely due to the slow rate of blood clearance of these liposomes which results in a strong background signal, particularly in the well-vascularized tissue of the lung. For the MAN-LIPs, the tumor-to-remote lung ratio was 2.4 times larger than plain, and 4.9 times higher than PEG liposomes. This is largely due to the rapid clearance of the MAN-LIPs from normal lung. The uptake by TAMs and rapid clearance from normal surrounding tissue is an important property for using the MAN-LIPs as a TAMs imaging agent.

### Discussion

Recent evidence has highlighted the role of macrophages, particularly of the M2 subtype, in the malignant progression of lung tumors. This recognition has spurred considerable effort to establish techniques to quantitatively monitor TAMs through noninvasive imaging. It has been previously shown that incorporating mannosylated phospholipids into liposomes improves targeting to peritoneal macrophages in vivo by receptor-mediated endocytosis. This approach for monitoring TAMs by PET imaging has two significant advantages over fluorescently-labeled iron oxide nanoparticles designed for detection by MRI and optical techniques. First, the targeted liposomes are radiolabeled and thus can be tracked using PET imaging, which possesses a higher intrinsic sensitivity (10⁻⁸ to 10⁻⁹ mol/L for PET compared to 10⁻³ to 10⁻⁵ mol/L for MRI). Second, liposomes provide a flexible platform for delivering both hydrophobic and hydrophilic cargo. For this study the aqueous interior core of liposomes was utilized to encapsulate a hydrophilic chelating agent to allow for remote labeling of the PET radionuclide, ⁶⁴Cu. This compartment of the liposome also could be used to encapsulate a wide array of hydrophilic drugs including cytotoxic agents that abolish TAMs through apoptosis or immunomodulatory agents that could trigger their reversal from a pro-tumoral M2 phenotype to a tumoricidal M1 phenotype. For example, it was recently shown that the cytokine IFN-γ induced human TAMs to switch from immunosuppressive to immunostimulatory phenotype.

Results from our in vitro studies revealed a significantly higher association of MAN-LIPs with M2-MØ compared with plain liposomes. Furthermore, M2-MØ exhibited significantly higher uptake of MAN-LIPs compared to M1-MØ, correlating with CD206 expression on these cells. Interestingly, M1-MØ exhibited a preference for MAN-LIPs over plain liposomes in spite of their low expression of CD206. This result may be explained by a study that found that IFN-γ enhances mannose receptor-mediated phagocytosis, despite its effects on surface mannose receptor down-regulation. More studies are needed to determine what receptors are responsible for the uptake of MAN-LIPs by M1-MØ and also if this uptake behavior occurs in vivo.

PET images showed enhanced MAN-LIPs signal in regions corresponding with lung tumors compared to areas of remote lung at 6 h post injection. Due to the small size of the lung tumors, we used a look-up table approach to estimate the RC value for each lung tumor in order to correct for PVE. We acknowledge some limitations to this approach. First, we did not account for radioactivity “spill-in” of surrounding tissue into tumor ROIs. We believe that the size of the lesion is the primary determinant of the RC, with background activity influencing it only to a small degree. This has been demonstrated by other groups with phantom experiments. Furthermore, as shown by in vivo and ex vivo PET images of the same lung, ROI-derived radioactivity concentrations of lung tumors were not significantly affected by their proximity to the liver. Another limitation of this approach is that we do not respiratory gated the PET images which can result in image blurring particularly in the thoracic region. Studies in mice have shown that non-respiratory gated PET imaging can lead to significant tumor standardized uptake value underestimations depending on lesion size. However, a gated PET acquisition typically suffers from a reduced signal-to-noise ratio compared to a non-gated one and because of this we chose not to employ it.

There were distinct differences between MAN and plain-liposomes in lung distribution following their co-injection. The fluorescence signal of plain liposomes was significantly higher than that of MAN-LIPs in areas of lung absent of visible tumors. This finding is likely not due to tissue autofluorescence because diD excitation occurs within a range of wavelengths where tissue autofluorescence is minimal. A possible explanation for the elevated remote lung signal is a delayed lung clearance of plain liposomes compared to MAN-LIPs. However, this is not likely since their measured blood clearance half-lives were nearly identical. Another possible explanation for this behavior could be due to an increased uptake of plain liposomes by resident alveolar macrophages. While this explanation is not consistent with other studies that have demonstrated enhanced targeting to alveolar macrophages by mannose-coated liposomes after intratracheal administration in rats and IV administration in mice, it is unknown if lung tumors influence the phagocytic behavior of these cells. Lastly, the complement system is a major factor in the clearance of liposomes. Additional studies would be needed to determine whether plain liposomes more efficiently activate complement, which could promote better recognition by alveolar macrophages and lead to higher lung sequestration. A flow cytometry study could be designed to help determine if lung resident macrophages are responsible for uptake or if the delayed lung clearance of plain liposomes is due to mechanisms not related to cellular uptake.

Our results obtained from confocal microscopy verified that macrophages were responsible for the elevated fluorescence signal observed within the areas of lung tumors on whole-lung fluorescent images. Microscopy revealed that all three liposome types were internalized by TAMs. The whole-lung fluorescence images of PEG-LIPs did not reveal high TME to background contrast due to the slow rate of clearance of these liposomes from the blood and prominent signal throughout the lung. These findings are significant because they indicate that MAN-LIPs, which are rapidly cleared from the blood relative to PEG liposomes, are able to target lung TAMs and that a slow rate of blood clearance is not required for targeting these cells. Rapid blood clearance and effective targeting are attractive properties of MAN-LIPs and highlight their potential use as TAM imaging and/or delivery agents. Our data show that MAN-LIPs not only demonstrated targeting of TAMs, but also rapid clearance in normal lung which is critical for achieving high tumor contrast by PET imaging.

### CONCLUSION

The influence of TAMs on tumor growth has been shown by numerous groups to be dependent on their differentiation state. In this paper we demonstrated the successful targeting of mannosylated liposomes to TAMs in a mouse model of pulmonary adenocarcinoma. Confocal microscopy verified that the PET signal was due to liposome internalization by TAMs. Although co-injection studies revealed that plain and PEG liposomes are both internalized by TAMs, MAN-LIPs exhibited the highest tumor to remote lung ratios on whole-lung fluorescent images showing that it is a promising agent for the localized delivery of imaging and potentially other agents to the tumor microenvironment.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

(1) Marin L, Colombo P, Beady M et al. Chronic obstructive pulmonary disease: patho-physiology, current methods of treatment and the potential for simvastatin in disease management. Expert Opinion on Drug Delivery. 2011; 8(9):1205-1220.
(2) Murphy B S, Bush H M, Sundareshan V et al. Characterization of macrophage activation states in patients with cystic fibrosis. Journal of Cystic Fibrosis. 2010; 9(5):314-322.
(3) Allavena P, Sica A, Solinas G et al. The inflammatory micro-environment in tumor progression: the role of tumor-associated macrophages. Critical Reviews in Oncology-Hematology. 2008; 66(1):1-9.
(4) Bingle L, Brown N J, Lewis C E, Bingle L, Brown N J, Lewis C E. The role of tumour-associated macrophages in tumour progression: implications for new anticancer therapies. Journal of Pathology. 2002; 196(3):254-265.
(5) Murdoch C, Muthana M, Coffelt S B et al. The role of myeloid cells in the promotion of tumour angiogenesis. Nature Reviews. 2008; Cancer. 8(8):618-631.
(6) Siveen K S, Kuttan G, Siveen K S, Kuttan G. Role of macrophages in tumour progression. Immunology Letters. 2009; 123(2):97-102.
(7) Robinson S C, Scott K A, Wilson J L et al. A chemokine receptor antagonist inhibits experimental breast tumor growth. Cancer Research. 2003; 63(23):8360-8365.
(8) Zeisberger S M, Odermatt B, Marty C et al. Clodronate-liposome-mediated depletion of tumour-associated macrophages: a new and highly effective antiangiogenic therapy approach. British Journal of Cancer. 2006; 95(3):272-281.
(9) Banciu M, Metselaar J M, Schiffelers R M et al. Antitumor activity of liposomal prednisolone phosphate depends on the presence of functional tumor-associated macrophages in tumor tissue. Neoplasia (New York). 2008; 10(2):108-117.
(10) Zhang B, Yao G, Zhang Y et al. M2-polarized tumor-associated macrophages are associated with poor prognoses resulting from accelerated lymphangiogenesis in lung adenocarcinoma. Clinics (Sao Paulo, Brazil). 2011; 66(11):1879-1886.
(11) Pollard J W, Pollard J W. Tumour-educated macrophages promote tumour progression and metastasis. Nature Reviews. 2004; Cancer. 4(1):71-78.
(12) Weissleder R, Kelly K, Sun E Y et al. Cell-specific targeting of nanoparticles by multivalent attachment of small molecules. Nature Biotechnology. 2005; 23(11):1418-1423.
(13) Leimgruber A, Berger C, Cortez-Retamozo V et al. Behavior of endogenous tumor-associated macrophages assessed in vivo using a functionalized nanoparticle. Neoplasia (New York). 2002; 11(5):459-468.
(14) Melancon M P, Lu W, Huang Q et al. Targeted imaging of tumor-associated M2 macrophages using a macromolecular contrast agent PG-Gd-NIR813. Biomaterials. 2010; 31(25):6567-6573.
(15) Allavena P, Chieppa M, Bianchi G et al. Engagement of the mannose receptor by tumoral mucins activates an immune suppressive phenotype in human tumor-associated macrophages. Clinical & Developmental Immunology. 2010; 2010:547179.
(16) Redente E F, Orlicky D J, Bouchard R J et al. Tumor signaling to the bone marrow changes the phenotype of monocytes and pulmonary macrophages during urethane-induced primary lung tumorigenesis in A/J mice. American Journal of Pathology. 2007; 170(2):693-708.
(17) Mantovani A, Sozzani S, Locati M et al. Macrophage polarization: tumor-associated macrophages as a paradigm for polarized M2 mononuclear phagocytes. Trends in Immunology. 2002; 23(11):549-555.
(18) Lee S J, Evers S, Roeder D et al. Mannose receptor-mediated regulation of serum glycoprotein homeostasis. Science. 2002; 295(5561):1898-1901.
(19) McGreal E P, Martinez-Pomares L, Gordon S, McGreal E P, Martinez-Pomares L, Gordon S. Divergent roles for C-type lectins expressed by cells of the innate immune system. Molecular Immunology. 2004; 41(11):1109-1121.
(20) Stathopoulos G T, Sherrill T P, Cheng D S et al. Epithelial NF-kappaB activation promotes urethane-induced lung carcinogenesis. Proceedings of the National Academy of Sciences of the United States of America. 2007; 104(47):18514-18519.
(21) Austyn J M, Gordon S, Austyn J M, Gordon S. F4/80, a monoclonal antibody directed specifically against the mouse macrophage. European Journal of Immunology. 1981; 11(10):805-815.
(22) Petersen A L, Binderup T, Rasmussen P et al. 64Cu loaded liposomes as positron emission tomography imaging agents. Biomaterials. 2011; 32(9):2334-2341.
(23) Yamazaki T, Nagata K, Kobayashi Y, Yamazaki T, Nagata K, Kobayashi Y. Cytokine production by M-CSF- and GM-CSF-induced mouse bone marrow-derived macrophages upon coculturing with late apoptotic cells. Cellular Immunology. 2008; 251(2):124-130.
(24) Kigerl K A, Gensel J C, Ankeny D P et al. Identification of two distinct macrophage subsets with divergent effects causing either neurotoxicity or regeneration in the injured mouse spinal cord. Journal of Neuroscience. 2009; 29(43):13435-13444.
(25) Mosser D M, Edwards J P, Mosser D M, Edwards J P. Exploring the full spectrum of macrophage activation. Nature Reviews. 2008; Immunology. 8(12):958-969.
(26) Litzinger D C, Huang L, Litzinger D C, Huang L. Amphipathic poly(ethylene glycol) 5000-stabilized dioleoylphosphatidylethanolamine liposomes accumulate in spleen. Biochimica et Biophysica Acta. 1992; 1127(3):249-254.
(27) Loening A M, Gambhir S S, Loening A M, Gambhir S S. AMIDE: a free software tool for multimodality medical image analysis. Molecular Imaging. 2003; 2(3):131-137.
(28) Matsui M, Shimizu Y, Kodera Y et al. Targeted delivery of oligomannose-coated liposome to the omental micrometastasis by peritoneal macrophages from patients with gastric cancer. Cancer Science. 2010; 101(7):1670-1677.
(29) Wijagkanalan W, Higuchi Y, Kawakami S et al. Enhanced anti-inflammation of inhaled dexamethasone palmitate using mannosylated liposomes in an endotoxin-induced lung inflammation model. Molecular Pharmacology. 2008; 74(5):1183-1192.
(30) Wijagkanalan W, Kawakami S, Takenaga M et al. Efficient targeting to alveolar macrophages by intratracheal administration of mannosylated liposomes in rats. Journal of Controlled Release. 2008; 125(2):121-130.
(31) Bauer A K, Cho H Y, Miller-Degraff L et al. Targeted deletion of Nrf2 reduces urethane-induced lung tumor development in mice. PLoS ONE [Electronic Resource]. 2011; 6(10):e26590.
(32) Redente E F, Dwyer-Nield L D, Merrick D T et al. Tumor progression stage and anatomical site regulate tumor-associated macrophage and bone marrow-derived monocyte polarization. American Journal of Pathology. 2010; 176(6):2972-2985.
(33) Shimkin M B, Stoner G D, Shimkin M B, Stoner G D. Lung tumors in mice: application to carcinogenesis bioassay. Advances in Cancer Research. 1975; 21:1-58.
(34) Kojima N, Biao L, Nakayama T et al. Oligomannose-coated liposomes as a therapeutic antigen-delivery and an adjuvant vehicle for induction of in vivo tumor immunity. Journal of Controlled Release. 2008; 129(1):26-32.
(35) Duluc D, Corvaisier M, Blanchard S et al. Interferon-gamma reverses the immunosuppressive and protumoral properties and prevents the generation of human tumor-associated macrophages. International Journal of Cancer. 2009; 125(2):367-373.
(36) Raveh D, Kruskal B A, Farland J et al. Th1 and Th2 cytokines cooperate to stimulate mannose-receptor-mediated phagocytosis. Journal of Leukocyte Biology. 1998; 64(1):108-113.
(37) Srinivas S M, Dhurairaj T, Basu S et al. A recovery coefficient method for partial volume correction of PET images. Annals of Nuclear Medicine. 2009; 23(4):341-348.
(38) Branco S S, Almeida P. Respiratory motion modeling in small animal PET using GATE. NSS '08. IEEE. Nuclear Science Symposium Conference Record. 2012. 2008.
(39) Singodia D, Verma A, Verma R K, Mishra P R. Investigations into an alternate approach to target mannose receptors on macrophages using 4-sulfated N-acetyl galactosamine more efficiently in comparison with mannose-decorated liposomes: An application in drug delivery. Nanomedicine: Nanotechnology, Biology and Medicine. In press.
(40) Ishida T, Harashima H, Kiwada H, Ishida T, Harashima H, Kiwada H. Liposome clearance. Bioscience Reports. 2002; 22(2):197-224.
(41) U.S. Patent Application Publication No. US 2007/0258993 A1, Apostolopoulos, et al., “DNA-Carrier Conjugate”, Nov. 8, 2007.
(42) U.S. Patent Application Publication No. US 2007/0292494 A1, Gieseler, et al., “Carbohydrate-Derivatized Liposomes for Targeting Cellular Carbohydrate Recognition Domains of CTL/CTLD/Lectins, and Intracellular Delivery of Therapeutically Active Compounds”, Dec. 20, 2007.
(43) U.S. Patent Application Publication No. US 2011/0262348 A1, Movahedi, et al., “Selective Targeting of Intratumoral Cells”, Oct. 27, 2011.
(44) U.S. Patent Application Publication No. US 2009/0215729 A1, Johnson, et al., “Microparticle Compositions to Modify Cancer Promoting Cells”, Aug. 27, 2009.
(45) Yihan Wu, et al., “Optical Imaging of Tumor Microenvironment”, Am. J. Nucl. Med. Mol. Imaging. 2013; 3(1):1-15.
(46) Makoto Matsui, et al., “Target delivery of oligomannose-coated liposome to the omental micrometastasis by peritoneal macrophages from patients with gastric cancer”, Cancer Science, July 2010, Vol. 101, No. 7, 1670-1677.
(47) Clara Kelly, et al., “Targeted Liposomal Drug Delivery to Monocytes and Macrophages”, Journal of Drug Delivery, Vol. 2011, Article ID 727241, 11 pages, doi:10.1155/2011/727241.

What is claimed is:

1. A method of detecting tumor associated macrophages comprising a mannose receptor, said method comprising contacting said tumor associated macrophages with a mannosylated liposome comprising a chelating agent, a detectable label, optionally a fluorescent dye, and optionally an additional therapeutic agent, subjecting said tumor associated macrophages to an imaging technique to detect said label, optionally imaging said fluorescent dye, and optionally quantifying said tumor associated macrophages contacted with said mannosylated liposome.

2. The method of claim 1 wherein said macrophages are associated with a tumor.

3. The method of claim 2, wherein said tumor is a cancer selected from the group consisting of squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, and head and neck cancer.

4. The method of claim 1, wherein said imaging technique is selected from the group consisting of fluorescence, positron emission tomography (PET), magnetic resonance imaging (MRI), single photon emission computed tomography (SPECT/CT), intravital laser scanning microscopy, endoscopy, and radiographic imaging.

5. The method of claim 1, wherein said detectable label is selected from the group consisting of a radionuclide, a radiological contrast agent, a paramagnetic ion, a metal, a biological tag, a fluorescent label, a chemiluminescent label, an ultrasound contrast agent and a photoactive agent.

6. The method of claim 1, wherein said chelating agent is selected from the group consisting of DTPA, DO3A, DOTA, EDTA, TETA, EHPG, HBED, NOTA, DOTMA, TETMA, PDTA, TTHA, LICAM, HYNIC, and MECAM.

7. The method of claim 1, wherein said mannose receptor is CD206.

8. The method of claim 1, wherein said liposome is remote loaded with said detectable label.

9. The method of claim 5, wherein said radionuclide is selected from the group consisting of ¹¹⁰In, ¹¹¹In, ¹⁷⁷Lu, ¹⁸F, ⁵²Fe, ⁶²Cu, ⁶⁴Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters.

10. The method of claim 9, wherein said label is ⁶⁴Cu.

11. The method of claim 1, wherein said tumor associated macrophage is an M2 macrophage.

12. The method of claim 1, wherein said wherein said method provides images of a tumor or the outline of a tumor.

13. The method of claim 1, wherein said mannosylated liposomes are taken up by said tumor associated macrophages.

14. The method of claim 13, wherein mannosylated liposome uptake is detected with PET.

15. The method of claim 14, wherein said uptake is quantified.

16. The method of claim 15, wherein said quantification is performed using A Medical Imaging Data Examiner (AMIDE) software and said quantification uses co-registered, resolution-matched magnetic resonance (MR) images to guide the size and location of PET regions of interest (ROIs).

17. The method of claim 16, wherein said mannosylated liposome uptake is quantified in tumor associated macrophages and in at least one additional tissue.

18. The method of claim 1, wherein said method is used to monitor the location of tumor associated macrophages.

19. The method of claim 1, wherein said liposome is an encapsulated liposome and comprises L-α-Phosphatidylcholine, cholesterol and optionally 3,3′-Dioctadecyloxacarbocyanine Perchlorate (DiO) and is made using a dehydration-rehydration process wherein 1,4,7,10-tetra-azacyclododecane-1,4,7,10-tetraacetic acid (DOTA) is added at the rehydration step, non-encapsulated DOTA is removed, said liposome is mannosylated by adding mannosylated phospholipid to the phosphatidylcholine and dissolving it in chloroform, wherein said mannosylated phospholipid was synthesized from mannotriose and dipalmitoylphosphatidylethanolamine (DPPE) by reductive amination, said detectable label is ⁶⁴Cu, said liposome is remote loaded with ⁶⁴Cu by ferrying ⁶⁴Cu into the liposome using a lipophilic transporter, mannosylated liposome uptake is detected with PET, said uptake is quantified using A Medical Imaging Data Examiner (AMIDE) software and said quantification uses co-registered, resolution-matched magnetic resonance (MR) images to guide the size and location of PET regions of interest (ROIs).

20. A mannosylated liposome for detecting a tumor associated macrophage, said mannosylated liposome comprising L-α-Phosphatidylcholine, cholesterol, optionally 3,3′-Dioctadecyloxacarbocyanine Perchlorate (DiO), 1,4,7,10-tetra-azacyclododecane-1,4,7,10-tetraacetic acid (DOTA), a detectable label, and optionally an additional therapeutic agent.

21. A method for making a mannosylated liposome for detecting a tumor-associated macrophage, said method comprising preparing an encapsulated liposome comprising L-α-Phosphatidylcholine, cholesterol and optionally 3,3′-Dioctadecyloxacarbocyanine Perchlorate (DiO) using a dehydration-rehydration process wherein 1,4,7,10-tetra-azacyclododecane-1,4,7,10-tetraacetic acid (DOTA) is added at the rehydration step, non-encapsulated DOTA is removed, said liposome is mannosylated by adding mannosylated phospholipid to the phosphatidylcholine and dissolving it in chloroform, wherein said mannosylated phospholipid was synthesized from mannotriose and dipalmitoylphosphatidylethanolamine (DPPE) by reductive amination, incorporating a detectable label and optionally an additional therapeutic agent.

22. The method of claim 1, wherein said method provides a greater tumor-to-tissue contrast ratio for imaging than using a non-mannosylated liposome.

23. The method of claim 22, where said contrast ratio is from about 2.0 to about 7.5.

24. The method of claim 1, wherein said macrophages are in a subject.
